(19) United States
(12) Patent Application Publication
(10) Pub. No.: US 2024/0024348 A1
(43) Pub. Date: Jan. 25, 2024

(54) METHODS AND COMPOSITIONS FOR THE TREATMENT OF GLAUCOMA AND RELATED CONDITIONS

(71) Applicant: Ophthalmic Therapeutic Innovation, Peabody, MA (US)

(72) Inventor: Guanting QIU, Peabody, MA (US)

(73) Assignee: Ophthalmic Therapeutic Innovation, Peabody, MA (US)

(21) Appl. No.: 18/356,874

(22) Filed: Jul. 21, 2023

Related U.S. Application Data
(60) Provisional application No. 63/391,303, filed on Jul. 21, 2022.
(60) Provisional application No. 63/491,070, filed on Mar. 19, 2023.

(51) Int. Cl. A61K 31/7076; A61K 9/00; A61P 27/06; A61K 31/472; A61K 31/498

(52) U.S. Cl. CPC A61K 31/7076; A61K 9/0048; A61K 9/0051; A61P 27/06; A61K 31/472; A61K 31/498

(57) ABSTRACT

Provided herein are compositions and formulations for alleviating problems associated with unregulated intraocular pressure (IOP) in an eye comprising the administration of Trabodenoson. Administration of Trabodenoson results in reversing or blocking disease progression of glaucoma and other related disorders. Trabodenoson also restores functionality of the pressure sensor in an eye.

### CROSS REFERENCE TO RELATED PATENT APPLICATIONS

This application claims benefit to U.S. Provisional Application No. 63/391,303, filed on Jul. 21, 2022, together with supplements filed on Jul. 30, 2022, Sep. 5, 2022, Oct. 1, 2022, Nov. 29, 2022, and Mar. 11, 2023; and U.S. Provisional Application No. 63/491,070 filed on Mar. 19, 2023, the entire contents of which are incorporated herein by reference.

### TECHNICAL FIELD

Embodiments of this disclosure relate generally to novel pharmaceutical compositions, formulations, dosing regimens, and to methods for making and using the same for treating glaucoma and related conditions. Provided herein are methods comprising the use of Trabodenoson for modulating and treating uncontrolled intraocular pressure.

### BACKGROUND OF THE INVENTION

Glaucoma is a multifactorial and slow progressive neural degenerative disease; it is the leading cause of irreversible blindness in the world with estimated of 118.5 million patients worldwide by 2025. While neural protection for glaucomatous vision loss is a widely sought after objective, there has yet to be any success with respect to effective outcomes. Intraocular pressure (IOP) is so far the only disease modifiable risk factor: controlling IOP enables the slowing down of disease progression and reduction of vision loss. There are over ten FDA approved IOP lowering drugs (including for example VYZULTA®, ROCKLATAN®, RHOPRESSA®), which are effective as symptom relief drugs in reducing the IOP in patients with early to mid-stage glaucoma. However, as glaucoma disease advances, patients become treatment non-responders (with the condition being referred to as refractory glaucoma), at which point, high risky invasive glaucoma drainage surgeries such as trabeculectomy or tube shunt become inevitable. Unfortunately, such procedures typically fail at a rate of 50% within 3-5 years for a variety of reasons, such as post-op surgical complications. Ultimately, about 25-30% glaucoma patients become legally blind because of uncontrolled IOP or IOP failure at the advanced or late stage of glaucoma.

The fundamental root cause of IOP failure or uncontrolled IOP is related to pressure sensor damage degradation which results in the failure of the pressure sensor to self-regulate the outflow through the trabecular meshwork (TM) in patients suffering from glaucoma or ocular hypertension (OH). At early to mid-stages of glaucoma, mild damage to the TM causes functional irregularities; at late stage glaucoma with severe damage in the TM causes engine breakdown. TM is responsible for 85-90% of the outflow drainage.

Traditionally, IOP lowering eye drops have been used as the mainstay glaucoma disease management (symptom relief) tool. Such eye drops work supplementing the deficits of pressure sensor irregularities at early to mid-stage glaucoma. Poor compliance by patients (>50% failure) causes IOP instabilities and fluctuations, which further accelerates the disease progression, ultimately becoming irreversible. A new generation of minimally invasive glaucoma surgeries (MIGS) address patient poor compliance, thus slowing down disease progression however they do not repair the pressure sensor pathological processes that occurs as a result of aging oxidative damage or para inflammation.

DURYSTA® is a bimatoprost sustained release (SR) biodegradable intracameral implant; it is the first and only FDA approved SR drug delivery product for the long-term management of IOP stability. DURYSTA® increases the levels of matrix metalloproteinase (MMP) in the ciliary muscles and iris then induces the decomposition of collagen, fibronectin and lamina within the extracellular matrix (ECM), resulting in improved uveoscleral outflow or possible trabecular meshwork outflow. DURYSTA® has led to a “drug holiday” in 80% of treated patients for at least 8 months, and up to 3 years in some individuals. DURYSTA®'s AMERTIS-II Phase 3 clinical trials further demonstrated the benefit of visual field stability over 20 months, including 8-month drug holiday period after 12-month API induction with three consecutive implants. DURYSTA® not only solves patient poor compliance, saves additional cost of medication, it also, importantly, stops glaucoma disease progression. Despite the promise of DURYSTA® however, its clinical application is limited by bimatoprost-triggered intraocular inflammation that oftentimes leads to cornea endothelium loss at an alarming speed (20% loss in less than 2 years vs 0.6% loss per year in normal condition). Such vision threatening side effects are proportional with its MMP therapeutic benefits in a time and dose dependent manner. Given the risk of such detrimental side effects, the FDA has allowed for single implant only, and no repeat applications. Single implant only led to 28% patients on drug holiday up to 20-month after 4-month API induction (Phase 2 clinical trial results).

What is needed are compositions and methods for alleviating complications caused by increased IOP. Ideally, such compositions and methods should be easy to administer, long lasting and safe. Furthermore, such compositions and methods should preferably be able to reverse and stop pathological states associated with IOP elevation in patients with glaucoma and related conditions. What is also needed are compositions that do not cause unwanted side effects.

### SUMMARY OF THE INVENTION

In an embodiment, the present disclosure relates to compositions and formulations comprising Trabodenoson as an IOP therapeutic agent (not simply as an IOP symptom relief agent) for patients with glaucoma including ocular hypertension (OHT), primary open-angle glaucoma (POAG), normal tension glaucoma (NTG), primary angle-closure glaucoma (PACG), secondary glaucoma as well as congenital glaucoma. In an embodiment, Trabodenoson restores the functionality of the pressure sensor in the trabecular meshwork (TM) in glaucomatous human eyes.

Though not wishing to be bound by the following theory, it is thought that Trabodenoson works by selectively binding to the adenosine A1 receptor (A1R) in the TM and ciliary body (CB), at an optimal dose which triggers three independent signaling pathways to regulate IOP. The action of Trabodenoson is particularly desirable in the situation where the TM is damaged or wherein the pressure sensor is worn out such that the pressure sensor therein displays functional irregularities (outflow engine broken or dysfunction). Trabodenoson regulates IOP via both slow mode and fast mode of action, the slow mode is through A1R activation at the TM cells resulting in the upregulation of the MMPs, including potent MMP-14 and MMP-2 at the TM. The MMP biological effect alters the texture of the TM tissue with an increased elasticity and hydro-conductance (similar to MMP on Bruch's membrane rejuvenation) by removing the ECM debris (e.g. type IV collagen, fibronectin, lamina) and opening up the mesh pore at aging rigid TM, thus improving oxygenation to the pressure sensing cell lining at the inner wall of Schlemm's canal. The pressure sensing cells are similar to the monolayer retinal pigment epithelium (RPE) having phagocytotic properties, derived from the neural crest with limited self-renewal ability upon injury in adult mammalian eyes. The lifespan and health of such pressure sensing cells determines the TM durability that regulates the outflow. The activation of MR also triggers a G-protein coupled cell membrane hyperpolarization that puts the TM endothelium cell (or pressure sensing cell) under a resting stage, reduces calcium influx, and restores the cell homeostasis (similar to its neural protection in the RGC cells), which has strategic importance to the TM health and longevity.

Both MMP induced basement membrane rejuvenation and adenosine triggered neural protection synergically improve the functionality of the pressure sensor in a diseased condition, and thus improve the IOP performance in patients with glaucoma. Use of Trabodenoson in a clinical setting has demonstrated continuous IOP improvement in a time course fashion: an aspect which has heretofore not been observed in association with other IOP lowering drugs for symptom relief. Surprisingly, such IOP pattern behavior is unique, robust and reproducible in early and advanced stage glaucoma, including PGA poor responders. The fast mode is considered to be via vascular effect, MR induced G protein coupled intracellular signaling has cross-talk with muscarinic receptor 2 (M2) at the contractible tissue in the TM and CB (mimic to pilocarpine effect), the pulsatile IOP synchronizes with the cardiac pulse, which has diurnal variance in both healthy and glaucoma diseased eyes. The fast mode of vascular effect shows a “bell” shape IOP dose response curve that requires an optimal dose or sweet spot in order to achieve a maximal IOP reduction. The pressure sensing cell is sensitive to oxidative damage and shear stress, and its functionality is nitric oxidate (NO) dependent or sensitive which in return determines Trabodenoson dosing optimal.

Provided herein are unique dosing regimens comprising the administration of Trabodenoson at clinically optimal doses. In certain embodiments, the clinically optimal dose of Trabodenoson is determined by its complex mechanism of action (fast vs slow mode). In an embodiment, single dose instillation of 0.6% eye drops of Trabodenoson results in a maximal IOP reduction and peaks at a “Bell” shape. In an embodiment, under the positive influence of MMP activation, an optimal dose for Trabodenoson eye drop comprises approximately 0.8%-6%, 1.0-3%, 1.0%-1.5%, or 1.2%-1.5%, or 1.5%. In an embodiment, for a sustained-release product, the API loading dose and release speed that results in an API concentration similar to that of the aqueous humor, comprises drop dosing (1.5%). The “bell” shape IOP lowering response curve is the guiding principle (rule) of dosing optimal for any products related to Trabodenoson for treating glaucoma (IOP) whilst attempting to achieve maximal IOP reduction.

IOP is regulated physiologically by a system of pressure sensing cells and its accompanying underlying basement membrane (BM) at the TM. The pressure sensing cells comprise a cobblestone type texture forming a monolayer lining at the Schlemm's canal inner wall, often referred to as TM endothelia cells, similar to RPE (retinal pigment epithelium) and cornea endothelium derived from the neural crest with limited self-renewal following injury or aging oxidative damage in adulthood human eyes. Episcleral vein (EVP) serves as the valve to regulate the outflow through the TM (pressure sensor).

In an embodiment of the invention, the novel dosing regimen described herein results in a drug holiday. A drug holiday for IOP related illness is achieved when the TM of the patient displays a healthy state with functional recovery of the pressure sensor to the extent that no medication or treatment is required to maintain the patient's IOP at the standard target level or under 21 mmHg. The invention as described herein surpasses the efficacy of DURYSTA® (bimatoprost sustained release intracameral implant) which leads to sustained drug holiday from 8 months up to 3 years. Though not wishing to be bound by the following theory, Trabodenoson is a more potent MMP stimulator (with an emphasis on MMP-14 as being the most potent among MMP family) with anti-inflammatory activity via activation of macrophage in oxidative condition, resulting in a longer drug holiday than DURYSTA®. Additionally, trabodensoon's cytoprotection or neural protection to the pressure sensing cells is unique attribute to its therapeutic effectiveness that is better than proinflammatory prostaglandin analogues (PGAs) in a SR system such as DURYSTA®.

Though not wishing to be bound by the following theory, it is thought that drug holiday induction time and durability is determined by two key aspects, first, stage of glaucoma disease and second, therapeutic potency and treatment duration. With regard to the stage of glaucoma disease, an important consideration depends upon the pressure sensor recoverability and durability or quality of the sensor. Based on existing evidence, it is thought that it may be necessary to have a minimal of 50-70% pressure sensing cells still present in glaucomatous eyes in order to achieve a full functional recovery and a stable drug holiday for 6-12 months or longer. Patients at the same disease staging may have different intricate cell biological competence, and accordingly, a given treatment may result in full recovery in some patients and a partial recovery in others, the duration could be from 3 months up to 3 years or longer, depending on the number of remaining pressure sensing cells and their metabolic state. With regard to the second aspect, therapeutic potency, this aspect is generally related to MMP enzymatic strength and neural protection potency. In contrast to prior art medications such as DURYSTA® which lack neural protection of pressure sensing cells (and in fact increase intraocular inflammation and metabolic stress (thereby compromising the health of pressure sensing cell) sustained release of trabodensoon is significantly more potent for treating glaucoma and related conditions. As described herein, sustained release of Trabodenoson is more effective than administration of other prior art medications especially as a drug holiday inducer. The longer induction time is given, the more stable of drug holiday may last. Furthermore, in comparison to prior art drugs such as DURYSTA®, sustained release of Trabodenoson can induce a drug holiday at a faster speed (e.g. 1-3 months) toward higher percentage of patients (80-90%) including more advanced stage of glaucoma (e.g. patients with 3 or 4 medications) with a stable and longer duration. For example, with 3-5-month sustained release of Trabodenoson, 50-80% patients with moderate glaucoma can achieve 6-24 months (or longer) of drug holiday respectively. Longer term sustained release administration drives a higher percentage of patients on a more durable drug holiday compared to shorter term sustained release format (e.g. 6M could be better than 3M for severe glaucoma). Though not wishing to be bound by the following theory, in comparison to Trabodenoson eye drops sustained release administration is more potent than the eye drops.

In an embodiment, the invention as envisioned herein, as either an eye-drop formulation or sustained release formulation (for example, as an implant), has superiority advantage over prior art formulations as Trabodenoson is the only drug in eye drop formula that can upregulate MMP, and the only MMP therapeutic agent that does not increase intraocular inflammation (prostaglandin analogue is proinflammatory), which makes Trabodenoson a prime choice and the best candidate for intraocular sustained release drug delivery product (such as anterior chamber rod). Trabodenoson is also a potent vascular dilator that has superiority therapeutic benefits to aging-related pathological processes involving with glaucoma and age-related macular degeneration (AMD).

### BRIEF DESCRIPTION OF FIGURES

FIG. 1 provides Table 2 showing data related to Trabodenson eye drops investigated in randomized controlled clinical trials undergoing dose escalation from Phase 1/2 to Phase 3 in patients with POAG/OH, including a combination study in Latanoprost eye drop poor responders. Total 819 patients or human subjects were tested for a period between 2 weeks to 3 months with doses starting from 0.15% BID to 6% QD and up to 18% single dose, which demonstrated excellent systemic clinical safety and ocular tolerability, conjunctiva hyperemia is low: 2.56% compared to 50% in Rhopressa (mild to moderate). The maximal IOP reduction is 6-7 mmHg by 1.5% BID at day 28 with possible switch to QD. Phase 3 clinical trials failed to meet the IOP endpoint at 3 months, but 6% QD arm showed an incremental increase of the IOP reduction (or improvement) over the course of 3 months with clinical significance compared to placebo (Data source: Clinicaltrial.gov).

FIG. 2 provides a graph demonstrating optimal dosing and showing Trabodenoson Eye Drop Dosing Optimal: 1.5% vs 0.6%. Phase 2 Trabodenoson dose escalation study showed a “Bell” shape IOP dose response curve with 0.6% BID peaked at the curve (Green bar) and a dose dependent IOP reduction with 1.5% BID being more effective than 0.6% BID at day 14 in normalizing the diurnal variance in patients with glaucoma (Blue Bar). This is the first time recognizing the two components pertinent to dual MOAs of Trabodenoson given at therapeutic dose (range). FIG. 2A: Shows single dose instillation of 0.6% Trabodenoson eye drop in Dutch-belted pigmented and normotensive rabbit eyes led to an instant IOP reduction of 25%-27% from the baseline at 2 hours and returned to the baseline at 6 hours. This study determines Trabodenoson dosing regimen that is twice daily at the start. FIG. 2B: Shows 14-Day Phase 2 clinical trials of Trabodenoson dose escalation in patients with POAG/OH demonstrated a “bell” shape dose response curve with 0.6% BID peaked at the top that is better than 0.15% BID and 0.3% BID on the left and 1.5% BID on the right (Green Bar). The Green Bar represents the IOP reduction from baseline IOP; the Blue Bar represents the IOP reduction from the diurnal IOP at day −1, for which the IOP response curve is a time and dose dependent with possible reaching to a steady state. 1.5% BID is more effective than 0.15% BID, 0.3% BID, 0.6% BID at Day 14. In cohort 1.5% BID, the mean IOP reduction at Day 28 is better than that at Day 14. Although 0.6% peaks at the Bell-shape dose response curve, under the positive influence of Trabodenoson derived MMP therapeutic, 1.5% BID is more effective than 0.6% BID in normalizing the diurnal variances in patients with glaucoma. FIG. 2 from T G Qiu. Trabodenoson on trabecular meshwork rejuvenation: a comprehensive review of clinical data. Expert Opin Investig Drugs. 2021 March; 30(3):227-236. doi: 10.1080/13543784.2021.1873276. Publisher: Informa Ltd and Taylor & Francis Groups. The intellectual property rights and copyright @ 2021 reserved by the corresponding author.

FIG. 3 provides diagrams demonstrating that Trabodenoson clinical dosing principles are governed by its dual mechanism of actions via a fast mode of vascular effect and a slow mode of MMP biological effect. The slow mode is through its derived MMP therapeutic leading toward a steady state in time, dose and disease staging dependent fashion (FIG. 3A). The fast mode showed a “Bell” shape dose response curve required a sweet spot or optimal dose in order to achieve the maximal IOP reduction, the IOP value is consistent with no changes over time (FIG. 3B). The clinical readout of the IOP reduction by Trabodenoson is the sum of fast and slow mode resulted IOP reduction, the maximal sum of the IOP reduction changes over time as the result of MMP therapeutic improvement that changes the IOP values (FIG. 3 C).

FIG. 4 provides the diagrams for Trabodenoson Dosing Optimal (1.5% vs 0.6%) & Time Course Dynamics (3 scenarios). Summary of dose selection: Under the positive influence of Trabodenoson derived MMP therapeutics, the previously identified optimal dose is no longer of 0.6% as the sweet spot at the start but shows a time course dynamic in a given disease staging. The dose selection is based on Trabodenoson MMP therapeutic potency leading toward a drug holiday (early-mid stage) and the need for quickly removing the ECM debris and preventing an irreversible damage to the pressure sensor (advanced or late stage). 1.5% is more effective than 0.6%. There are three different clinical scenarios illustrating the 0.6% BID and its next dose of 1.5% BID in tracing each other towards the finishing line (steady state) where drug holiday becomes possible. FIG. 4A: The first scenario illustrates that 1.5% BID reaches to the steady state (drug holiday) at an early timepoint (e.g. between 4-8 months), and 0.6% BID is still running (ramping up), has not yet achieved the steady state, the sum of the IOP reduction in 1.5% BID is higher than that with 0.6% BID in a given group of patients. 1.5% is more effective than 0.6% in treating patients with severe glaucoma. FIG. 4B: The second scenario illustrates that 0.6% BID reaches to the steady state (drug holiday) at a later time point (e.g. 8-12 months), and the sum of its IOP reduction could be higher than that with 1.5% BID which has reached to a steady state at the earlier time with a stable IOP. FIG. 4C: The third scenario illustrates 1.5% BID and 0.6% BID having a transit overlap, for which the sum of IOP reduction in both doses are the same or similar for a very short time, this transit moment could be at an early time point (e.g. 1-4 months) before both doses reach to the steady state, when IOP in 1.5% is ramping up with the MMP effect whereas 0.6% vascular effects surpass 1.5% with an advantage. It also could be at a late time point (e.g. 6-9 months) after 1.5% BID achieves the drug holiday with a stable IOP (flat curve) and the IOP with 0.6% BID is still ramping up to the extent that may surpass the maximal value rendered by 1.5% BID, during this dynamics, there will be a touching point where both doses reach to the same sum of IOP reduction before 0.6% BID reaches to the steady state (drug holiday). Of note, the timepoints 4-8 months, 8-12 months and 1-3 months or 6-9 months are given for illustration purposes, does not reflect the actual time in a real world in patients. The dose frequency BID may be switched to QD after a period of time for both doses, but for comparative purpose in this illustration, BID will be used consistently throughout. Eye drops and sustained release (SR) share the same dynamic pattern: 1.5% BID is more effective than 0.6% BID in reaching to the steady state (or drug holiday) especially in treating severe glaucoma. Sustained release (SR) formula is more effective than its eye drops formula (at the same API concentration in the aqueous humor). The timeline for patient achieving a drug holiday (steady state) depends on the formula (SR vs eye drops) and disease severities, although 0.6% BID may achieve a slightly higher of the IOP reduction (in sum) at a later time, from therapeutic point of view, 1.5% is more effective and will be quicker to achieve a drug holiday, hence 1.5% is better than 0.6% as an IOP therapeutic agent to glaucoma patients, especially the severe disease that requires immediate repair of the pressure sensor before its reaching to an irreversible cell apoptosis (cell loss) to the extend exceeding the threshold (not known) of its functional compensation. Based on the preclinical and clinical evidence (FIG. 8), 6% perhaps is at the bottom of the Bell shape IOP dose response curve, and 3% falls at the half-way down the slope showed in this diagram. Trabodenoson induced neurovascular modulation is NO sensitive or dependent.

FIG. 5 provides data related to studies demonstrating Trabodenoson Unique IOP Pattern Behaviors in Patients with POAG/OH. This panel demonstrated a unique and potent IOP therapeutic improvement in patients with early to moderate POAG/OH including PGA poor responders, such unique pattern behavior sets Trabodenoson apart from currently FDA approved IOP drugs, such as ROCK inhibitor, Rhopressa, PGAs (lantanoprost, bimatoprost, travoprost, vyzulta), beta blockers, CAI, and Brimonidine, which almost all are of symptomatic relief demonstrating a flat IOP lowering curve in a time course fashion, with Vyzulta and Rescula exhibiting some mild and limited therapeutic benefits but in a subset group (NTG) or selective individuals, respectively (FIGS. 5E and 5F). The unique time course IOP improvement of Trabodenoson is the result of its MMP therapeutic and neural protection to the pressure sensing cells via TM rejuvenation and repair of the TM pressure sensor, which is the first time revealed in this invention. The IOP profile of latanoprost, travoprost, bimatoprost, unoprostone and rhopressa can be found in randomized controlled clinical trials in references of AS Khouri et al 2019, ES Ancient et al. 2005, DR Fung et al 2014, commonly known as a flat curve in a time course over 6 to 12 months or longer in patients with POAG/OH. FIG. 5A: Phase 3 Trabodenoson monotherapy in patients with mild POAG/OH at baseline IOP<=24 mmHg given at 6% QD high dose resulted in a continuous IOP improvement over a course of 3 months with clinical significance compared to placebo (FIG. 5A from TG Qiu. Expert Opin Investig Drugs. 2021 March; 30(3):227-236. Publisher: Informa Ltd and Taylor & Francis Groups. The intellectual property rights and copyright @ 2021 reserved by the corresponding author). FIG. 5B provides data related to: Phase 2 Trabodenoson dose escalation study in patients with POAG/OH at baseline IOP>21 mmHg showed an incremental increase of IOP reduction (improvement) by 1.5% BID eye drops, day 28 performed better than Day 14 (improved 0.9 mmHg in 2 weeks), BID dosing switch occurs as early as day 29. (FIG. 5B from Nilyers J S, et al. Journal of Ocular Pharmacology and Therapeutics 2016 32:8, 555-562. https://www.liebertpub.com/doi/10.1089/jop.2015.0148. Copyright @ 2016, Authors and Mary Ann Liebert, Inc Publishers) FIG. 5C: Trabodenoson add-on to Lantanoprost in POAG/OH patients with Latanoprost poor responders also showed a robust and continuous IOP improvement over the course of 12 weeks with dosing regimen of 1.5% BID for 8 weeks, then switching to 3.0% QD for another 4 weeks. All above three clinical trials (Ph2 and Ph3) have demonstrated the same unique IOP pattern behaviors (IOP improvement) in a time course fashion with possible dosing switch from BID to QD, indicating patient's TM durability improved after Trabodenoson treatment (FIG. 5C from Inotek Pharma IPO S-1 form, 2014). FIG. 5D showed DURYSTA® derived MMP therapeutic in preclinical normotensive dog model, its IOP lowering profile is a dose dependent with 22 ug being better than 12 ug API loading dose, however, there is no time course improvement. In clinical trials (not showed), there is also no time course improvement, three possible reasons, one may be related to its increased intraocular inflammation that comprises the IOP performance, secondly, DURYSTA® does not have neural protection benefit to the pressure sensing cells or TM endothelium. Thirdly, SR quickly reached to a steady state within the first week or two. (Source: Lee S S, et al. J Ocul Pharmacol Ther. 2019 April; 35(3):138-144. doi: 10.1089/jop.2018.0095.) FIG. 5E. Vyzulta (Latanoprost Bunod) IOP reduction profile over 12-month (12 M) in patient with NTG showed NO donation benefit with slight improvement (˜0.6 mmmHg) within the first 12-week, then the curve flattened out for the rest of the 12 M study (FIG. 5E from Kawase, K. et al. Adv Ther 33, 1612-1627 (2016). Copyright @ 2016 Authors. https://link.springer.com/article/10.1007/s12325-016-0385-7#rightslink.) FIG. 5F showed an example of Rescula eye drop induced IOP “therapeutic” profile in two individual patients with refractory glaucoma, suggesting a slow mode and accumulative improvement over time from weeks or months depending on the disease severity, no drug holiday was ever reported in Rescula user. Rescula given to early or moderate glaucoma patients, or patients who does not have “active” intraocular inflammation, its IOP lowering profile showed a flat curve, similar to the symptom relief drugs (Ref: E S Arcieri et al. Arch Ophthalmol. 2005; 123(2):186-192). FIG. 5F from Qiu TG (2015) J Clin Exp Ophthalmol 6: 473 Copyright @ Authors 2015).

FIG. 6 provides data related to studies showing Trabodenoson Therapeutic Effectiveness in Favor of Severe Glaucoma and Conditions. As shown herein, Trabodenoson is more effective in patients with more severe glaucoma compared to those with less severe conditions, more effective in POAG than in OH. The IOP improves with treatment time in both mild and advanced stage POAH/OH. FIG. 6A: Phase 2 clinical trial showed Trabodenoson eye drops given 1.5% BID resulted higher IOP reduction in patients with POAG than that with OH in early-stage population. (FIG. 6A from Qiu TG. Expert Opin Investig Drugs. 2021 March; 30(3):227-236, Copyright @ 2021 Authors) FIG. 6B: Ph2 clinical trials showed that Trabodenoson eye drops given at 1.5% BID resulted to higher IOP reduction in patients with baseline IOP>25 mmHg than those with baseline IOP>21 mmHg (>7 mmHg vs 6.5 mmHg) at day 14, and the IOP improves over treatment time with day 28 being better than day 14 (Data source: Inotek Pharma OIS 2012). FIG. 6C: Phase 2 clinical trials of Trabodenoson add-on to patients who are Lantanoprost poor responders showed a robust and incremental IOP improvement over a course of 12-week treatment time, with dosing switch from 1.5% BID*8 weeks to 3% QD*4 weeks. Furthermore, Trabodenoson is more effective in patients with POAG than with OH in Lantanoprost poor responders (data source: Inotek Pharma 2014 S-1 Form).

FIG. 7 provides data related to Trabodenoson Therapeutics on Pressure Sensor via Neural Protection and ECM Rejuvenation at TM. As shown herein, Trabodenoson treatment provides neural protection to the pressure sensing cells (neuronal lineage cell), at the same time it removes the ECM debris through upregulations of potent MMP-14 subsequently leading to the degradations of collagen IV and fibronectin at the thickening ECM of trabecular meshwork in glaucomatous eyes, both mechanism of actions synergistically led to rejuvenation and repair of the pressure sensor, which has not been understood or reported before. FIG. 7A: In preclinical NAION mice model, Trabodenoson given at 6% eye drops BID for a period of time, the EM morphological evaluation on the across-section of the optic nerve bundle demonstrated a significant reduction of the axon loss and myelination damage observed in the treated eyes compared with placebo control. TM endothelium is similar to RPE, cobblestone alike, monolayer with self-contact inhibition in cell culture system; both derived from the neural crest and lost self-renew capacity in adult mammalian eyes (W D Stamer et al, Exp. Eye. Res. 2017 May; 158:112-123). MR activation induced neural modulation is inhibitory by nature, through its G protein coupled cell membrane hyperpolarization leading to neuronal cell towards a resting stage by increasing the K-ATP conductance, inhibiting calcium influx and excitatory neural transmitter release such as glutamate and dopamine. The evidence of Trabodenoson on RGC neural protection is transferrable to its cytoprotection to the pressure sensing cells at the TM; by putting these cells under a resting stage is essential for replenishing the cell metabolic competence (rejuvenation) (FIG. 7A from Yan Guo, et al; Trans. Vis. Sci. Tech. 2019; 8(6):47, doi: https://doi.org/10.1167/tvst.8.6.47. Copyright @2019 Authors, published by ARVO Journal). FIG. 7B/C: In vitro cultural human TM cell model, Trabodenoson demonstrates its biological activities of upregulating MMP-14 expression, subsequently resulted in downregulation of collagen IV and fibronectin. FIG. 7D: In preclinical aged mice model, 6% Trabodenoson treatment led to TM mesh pores opening up and ECM remodeling with enlarged ECM space, compared to placebo treated eyes (FIG. 7B/C/D source: Li G, Stamer W D et al. Trabodenoson, an Adenosine Mimetic With A1 Receptor Selectivity Lowers Intraocular Pressure by Increasing Conventional Outflow Facility in Mice. Invest Ophthalmol Vis Sci. 2018 Jan. 1; 59(1):383-392. https://10.1167/jovs.17-23212. Copyrights @ 2018 Authors, published by ARVO Journal). TM is a sponge-like tissue composing of ECM, the outflow resistance lies at the JCT transitioning to the basement membrane (BM) underlying the monolayer endothelium that forms the inner wall of Schlemm's canal. The BM is made of fibronectin, lamina and collagen IV in healthy eye, in glaucomatous eyes, there are pathological ECM materials with deformed proteoglycan depositing on the BM and clotting the TM mesh pores. Trabodenoson derived MMP-14 treatment removes the ECM debris, opening up the mesh pores, and leading to the texture changes of ECM with increased elasticity and hydraulic conductance, similar to MMP-9 treatment effects on the Bruch's membrane in retina (FIG. 9). The CHA (A1R) derived MMP therapeutic effects on TM rejuvenation was first discovered by Dr Qiu in 2015. Trabodenoson is a more potent MMP stimulator with MMP-14 being the most potent MMP in the family. Its therapeutic potency in the aged mice is in concert with that in patients of severe condition.

FIG. 8: Trabodenoson 3% QD is better than 6% QD on the “bell” shape IOP dose reduction curve, a fast mode via vascular effect. MR on vascular effect is through Gi-proteins coupled muscarinic M2 neuro-vascular modulation by increasing nitric oxide (NO) effect, similar to the Acetylcholine (Ach) effect on muscle relaxation with increased NO, MR acts through Gi-protein activation coupling with cAMP production that causes vascular relaxation (dilation) with Alit mediated NO production. The targeted receptor is perhaps through M2 parasympathetic nerve innervation on the blood vessel or contractible tissue such as TM smooth muscle cells, and TM endothelium cell which is most sensitive to NO. FIG. 8A/B: Trabodenoson eye drops was given at 3% QD and 6% QD in young and aged C57 mice for 7 consecutive days respectively, at study day 1, Trabodenoson 6% QD seems more effective in aged mice than that in young mice given at 3% QD, which is in concert with clinical study in patients as the result of MMP effects. It is also noticeable that the IOP reduction in 3% QD sustained over 7 days whereas its IOP lowering effect in 6% QD aged mice slowly diminished at day 7, probably the 6% QD falls at the bottom of the bell shape IOP dose response curve. Mouse eye has a very thin sclera that increases drug delivery efficiency to the pars plana and vitreous cavity (small), which causes accumulative cAMP level (high) and exceeds the optimal NO level. Trabodenoson on vascular effect is dose sensitive. This study result was not properly interpretated in prior publication, this is first time noted that Trabodenoson dosing in IOP reduction is possibly related to the NO effects. 3% is better than 6% in IOP reduction in a mice model. Similar findings were seen in patients (FIG. 8A/B from Li G, W D Stamer et al. Invest Ophthalmol Vis Sci. 2018 Jan. 1; 59(1):383-392, doi: 10.1167/iovs.17-23212. Copyright @ 2018 Authors, published by ARVO Journals). FIG. 8C: In Phase 2 Clinical Study of Trabodenoson Fixed Dose Combination with Lantanoprost, 3% QD Trabodenoson also works better than 6% QD in combination with 0.005% QD Lantanoprost, but both seem less effective than 0.005% QD Lantanoprost alone, possibly due to the conflicts of PGA and Alit on vascular effects for one being a dilator and the other being vascular constrictive (Source: Inotek Pharmaceuticals, Clinical Trial Identifier: NCT02829996).

FIG. 9 provides demonstration of MMPs on Bruch's membrane rejuvenation. FIG. 9A/B show that MMP-2 increases hydraulic conductance of aging Bruch's membrane from human donor eyes. MMP-9 is more potent than MMP-2, its treatment shifts the curve of Bruch's membrane hydraulic conductance in 60-year-old donor tissue to regain 40-year-old's Bruch's membrane's hydraulic conductance with increased elasticity (FIG. 9A/B from Ahir A, Guo L, Hussain A A, Marshall J. Expression of metalloproteinases from human retinal pigment epithelial cells and their effects on the hydraulic conductivity of Bruch's membrane. Invest Ophthalmol Vis Sci. 2002 February; 43(2):458-65. Copyright @ 2002 ARVO publisher). FIG. 9C shows that nano pulse laser stimulates host RPE cell regeneration (limited) with increased MMP production at the Bruch's membrane which resulted in subretinal fluid resolution in patients with DME, the clinical efficacy demonstrates a slow accumulative mode with sustained therapeutic benefits (vision gain). This is the MMP therapeutic pattern behavior in retinal specialty, similar to what was seen in Trabodenoson derived MMP therapeutic outcome of IOP improvement (slow accumulative mode). Connecting dots from retinal to glaucoma, MMP and basement membrane (BM) is the key common string. Bruch's membrane is a type of BM. (FIG. 9C from Qiu TG. Expert Opin Investig Drugs. 2021 March; 30(3):227-236. Copyrights @ 2021 Authors)

FIG. 10 demonstrates that DURYSTA® derived MMP leading to Drug Holiday with Functional Recovery of the Pressure Sensor in Patients with Mild to Moderate Glaucoma, however the holiday period was unstable, possibly due to the increased intraocular inflammation, proportionally driven by high level SR API released to in the AC. FIG. 10A: Ph2 clinical trial: DURYSTA® with 4.2 month API release led to 68%, 40%, and 28% patients with mild to moderate POAG/OH on drug holiday for 2 months, 8 months, 20 months respectively, its MOA is believed to be through high concentration Bimatoprost triggered MMP upregulation (e.g. MMP-9) leading to ECM remodeling and TM rejuvenation toward a full functional recovery of the pressure sensor (FIG. 10A from Craven E R, et al. Drugs. 2020 February; 80(2):167-179; https://link.springer.com/article/10.1007/s40265-019-01248-0#rightslink. Copyright @ 2019 Authors published by Springer Nature). FIG. 10B: In vitro human TM cell cultural model, a high concentration (10 ug/mL) of Bimatoprost and Latanoprost upregulates MMP-9 expression (FIG. 10B from Li X, et al. PLoS One. 2016 Mar. 24; 11(3):e0151644, doi: 10.1371/gournal.pone.0151644. Copyright @ Authors 2016). FIG. 10C: Elevated cytokine and proinflammatory molecules (e.g. TNFa, Interleukins) in the aqueous humor in patients with uveitis, diabetic retinopathy and others led to cornea endothelium loss (CEL) with clinical significance, which shows a dose dependent or disease severity dependent linear fashion. (FIG. 10C from Yagi-Yaguchi, Y et al. Sci Rep 7, 13603 (2017). https://doi.org/10.1038/s41598-017-14131-3. Copyright @ 2017 Authors, published by Springer Nature).

FIG. 11 and FIG. 12: Trabodenoson on Drug Holiday Parameter Estimates. Scientific Basis: Drug holiday reflects the state of TM health and a full functional recovery of the pressure sensor in glaucomatous eyes. It reflects the intricate biological competence of pressure sensing cells that can self-regulate the IOP without the need of extra help of symptom relief IOP medications. Drug holiday parameter is determined by two key aspects: one aspect is related to glaucoma disease staging defines the pressure sensor recoverability (estimated: it needs to have minimal 50-70% pressure sensing cell remained in glaucomatous eyes in order to achieve a full functional recovery and a stable drug holiday for 6-12 months or longer. Parameters should include the percentage of patients: e.g. 50%-80%, time to start (1-3 month or 3-6 month induction), and the durability of holiday period (3-6 M or 12-24 M or 3-5 years). The other aspect is related to therapeutic potency and treatment time. Potency includes MMP potency and neural protection potency, Durysta does not have neural protection role to the pressure sensing cell, plus it increases inflammation and metabolic stress to the pressure sensing cells. Therefore, Trabodensoon is far more potent than PGA in SR such as Durysta. Therapeutic benefits of Trabodenoson is increased with treatment time, hence its SR is more effective than the eye drops as drug holiday inducer. High concentration eye drop is more effective than low concentration eye drops, e.g. 1.5% is more effective than 0.6% as drug holiday inducer. 3% is therapeutically more effective than 1.5% as drug holiday stabilizer without the need of maximal target of IOP reduction.

In principle, Trabo-SR (sustained release Trabodenoson) format can induce a drug holiday at a faster speed, at higher percentage of patients including more advanced stage of glaucoma (e.g. patients with 3-4 medications or glaucomatous eyes received trabeculectomy or IDose implant but failed) with a longer duration compared to Durysta. Also, to Trabodenoson, longer SR induction drives higher percentage patients on a more durable drug holiday compared to shorter term SR format (e.g. 6 M is better than 3 M for severe glaucoma). However, Durysta MMP therapeutic benefit is proportionally increased with its sight threatening side effect to the cornea and TM health, hence, its MMP benefit is being compromised.

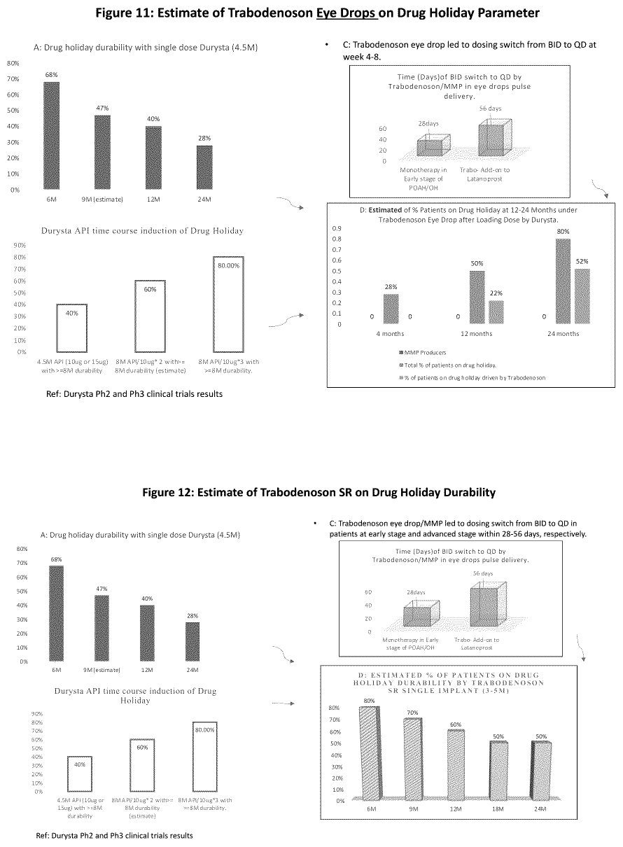

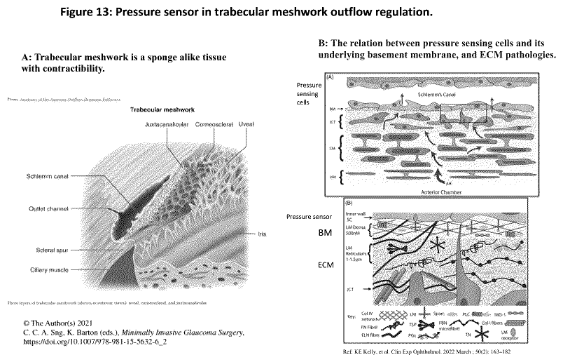

Clinical Evidence: FIG. 11-12A: Drug holiday durability with single dose implant of Durysta SR (4.2 M loading API of bug and 15 ug) has led to estimate of 68%, 47% (estimated), 40%, and 28% patients on drug holiday at 6 M, 9 M, 12 M, and 2 M respectively. FIG. 11-12B: Durysta API time course induction of drug holiday: Durysta SR single implant with 4.2 M API loaded with bug and 15 ug led to 40% on drug holiday at 12 M, and 12 month API with 3 consecutive implant of bug led to 80% patients on drug holiday. Estimated about 60% patients on drug holiday by 8-month API (1 or 2 Trabodenoson implants). FIG. 11-12C: Trabodenoson given at low dose eye drops (1.5% BID) led to unique dosing switch from BID to QD as early as day 29 in mild-moderate POAG/OH, and 8 weeks in Latanoprost poor responders. The dosing switch is a clinical biomarker of the TM durability, a precursor of drug holiday.

Real world possibilities: FIG. 11D provides data showing an estimate of Trabodenoson eye drops administration on drug holiday durability: Estimated drug holiday of Trabodenoson eye drop in patient with glaucoma when combined with a SR MMP as a loading dose (e.g. DURYSTA®) in the relay: 50% patients at 12 months or 80% patients at 24 months could achieve a durable drug holiday for minimal 6 months up to 3 years. Trabodenoson eye drops also can stabilize and prolong the drug holiday up to 12 months or longer in Patients with Durysta SR MMP induction (67% holiday induction by Durysta but 27% were short lived for less than 2 months). FIG. 12D shows estimated drug holiday of Trabodenoson SR (AC rod) in patients with glaucoma as a holiday inducer with 3-5-month API, it is estimated of 50-80% patients with 1-2 meds can achieve a stable drug holiday for 6-24-month holiday durability respectively. 2-3 M API might induce drug holiday in newly diagnosed early stage glaucoma.

FIG. 13A illustrates trabecular meshwork as a sponge alike tissue, composing of contractible tissues, forming the outflow drainage system with Schlemm's canal outlet the aqueous flow toward episcleral vein, and episcleral venous (EVP) serves as the valve of the IOP regulator, which is governed by the pressure sensor (FIG. 13A from Kay Lam, et al. Anatomy of the Aqueous Outflow Drainage Pathways. Book title: Minimally Invasive Glaucoma Surgery (pp. 11-19). January 2021. Copyright: the authors @ C. C. A. Sng, K. Barton). FIG. 13B: illustrates the pressure sensing cells forming monolayer lining at the inner wall of SC and its underlying BM, which forms functionally and anatomically integral part of the pressure sensor. The ECM in the JCT also plays a key role along with the BM to maintain and support the metabolic health and functionality of the pressure sensing cells. The outflow resistance in TM is believed to be at JCT region. MMP therapeutic helps to clean up the ECM debris and pathological proteoglycans at the dense JCT region with an improved elasticity and hydraulic conductance, subsequently increases the oxygenation of the pressure sensing cells which rejuvenate its metabolic competence, contributing to the sensor's longevity. Trabodenoson induced neural protection to the pressure sensing cell by putting the cell under a resting stage is unique and important to prevent the cell from apoptosis or necrosis under oxidative stress or metabolic inflammation. (FIG. 13B from Keller K E, Peters D M. Pathogenesis of glaucoma: Extracellular matrix dysfunction in the trabecular meshwork-A review. Clin Exp Ophthalmol. 2022 March; 50(2):163-182).

All the cited Figures published at open access journals, covered by the Creative Commons Attribution-NonCommercial-NoDerivatives 4.0 International License (http://creativecommons.org/licenses/by-nc/4.0/), the copyrights have been granted at the courtesy either by the corresponding author(s) or publishers.

### DETAILED DESCRIPTION

The following detailed description is exemplary and explanatory and is intended to provide further explanation of the present disclosure described herein. Other advantages, and novel features will be readily apparent to those skilled in the art from the following detailed description of the present disclosure. Texts and references mentioned herein are incorporated in their entirety, including United States Provisional Patent Application Nos. 63/491,070, and No. 63/391,303.

Also incorporated herein in their entirety are U.S. Pat. Nos. 7,423,144, 8,501,708, 9,522,160, 9,370,530 and US Patent Application Serial No. US2016/0158268.

As used herein, the term “subject” should be construed to include subjects, for example medical or surgical subjects, such as humans and other animals requiring therapeutic intervention.

### Abbreviations

MR: adenosine A1 receptor
AAV: adeno-associated virus
AC: anterior chamber
Ach: acetylcholine (Ach)
ADON: autosomal dominant optic neuropathy
AMD: age-related macular degeneration
ARTEMIS 1: Phase 3, Randomized, 20-Month Study of Bimatoprost Implant in Open-Angle
Glaucoma and Ocular Hypertension.
ARTEMIS 2: Phase 3, Randomized, 20-Month Study of the Efficacy and Safety of Bimatoprost
Implant in Patients with Open-Angle Glaucoma and Ocular Hypertension.
API: active pharmaceutical ingredient
ARVO: Association for Research in Vision and Ophthalmology.
BID: twice a day
BM: Bruch's membrane
BM: basement membrane
C3: complement factor 3
CB: ciliary body
CEL: cornea endothelium loss
CHA: cycloheylendenosine
CFTR: Cystic fibrosis transmembrane conductance regulator
CM: corneoscleral meshwork
DME: diabetic macular edema
ECD: endothelial cell density
ECM: extracellular matrix
ERK: extracellular signal-regulated kinase
EVP: episcleral veinous pressure
GA: geographic atrophy
IND: investigational new drug
IOP: intraocular pressure
IVT: intravitreal
JCT: juxtacanalicular tissue
LBN: latanoprost bunod
LCA: Leber's congenital amaurosis
LHON: Leber's hereditary optic neuropathy
MATrX-1: Study of Trabodenoson in Adults With Ocular Hypertension or Primary Open-angle
Glaucoma.
MIGS: minimally invasive glaucoma surgery
MMP: matrix metalloproteinase
MOA: mechanism of action
6 M: 6 months or 6-month.
MS: multiple sclerosis
mTOR: mechanistic target of rapamycin
NAION: non-arteric anterior ischemic optic neuropathy
NO: nitric oxide
NTG: normal tension glaucoma
OH: ocular hypertension
PACG: primary angle-closure glaucoma
PGA: prostaglandin analogue
POAG: primary open-angle glaucoma
POC: proof of concept
PXF: Pseudoexfoliation
QD: once a day.
RD: retinal detachment
RGC: retinal ganglion cells
ROCKi: Rho kinase inhibitor
RP: retinitis pigmentosa
RPE: retinal pigment epithelium
2RT laser: retinal rejuvenation (2RT) laser,
SC: Schlemm's canal
SLT: selective laser trabeculoplasty
SR: sustained release
TID: three times a day.
TM: trabecular meshwork
TNF: tumor necrosis factor
UM: uveoscleral meshwork
VEGF: vascular endothelial growth factor

In an embodiment, provided herein are novel methods and dosing regimens for improving intraocular pressure comprising the administration of a composition comprising Trabodenoson. The chemical structure of Trabodenoson (formerly known as INO-8775) is provided below:

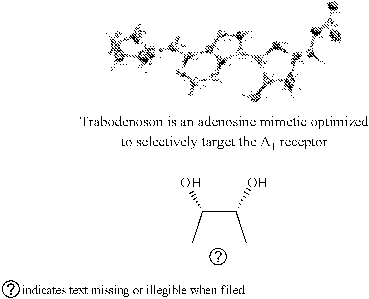

Though not wishing to be bound by the following theory, it is thought that Trabodenoson acts by binding to adenosine A1 receptors. A1 receptors are distributed in ocular tissues as shown below:

TABLE 1
Adenosine A1 Receptor Distribution in Ocular Tissues.

| A1 receptor distribution | Monkey | Human |
|---|---|---|
| Cornea Epithelium | (+++) | |
| Cornea endothelium | (+++) | |
| Trabecular Meshwork | (++) | (+++) |
| Iris Sphincter | (+++) | |
| Ciliary Muscle | (+++) | |
| Ciliary Epithelium | (+++) | |
| Retinal Ganglion Cells | (+++) | (+++) |
| Optic Nerve Fiber | (+++) | (+++) |
| RPE | (+/−) | (+/−) |
| Muller's Glial Cell | (+++) | (+++) |
| Microglial | (++) | (++) |
| Photoreceptor Inner segment (PR- | (++) | (+) |
| Outer Plexiform Layer (OPL) | (++) | (+) |
| Vascular endothelium | | (+++) |

Ref 1: K. M. Beach et al. Exp Eye Res. 2018 September; 174: 40-50.
Ref 2: K. M. BRAAS, MA. ZARBIN, and S. SNYDER. Proc. Natl. Acad. Sci. USA. 1987, Vol. 84, pp. 3906-3910.

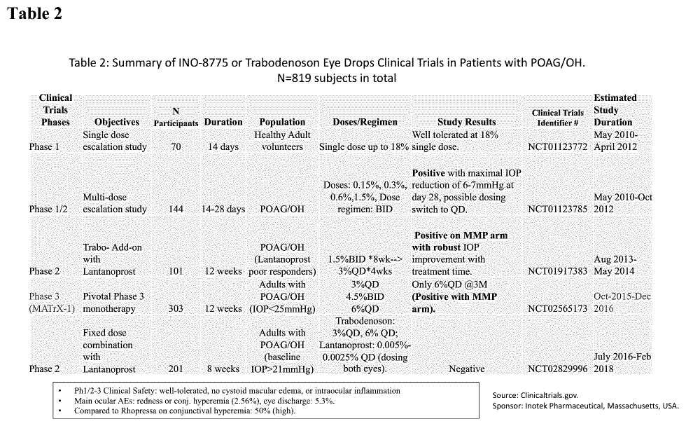

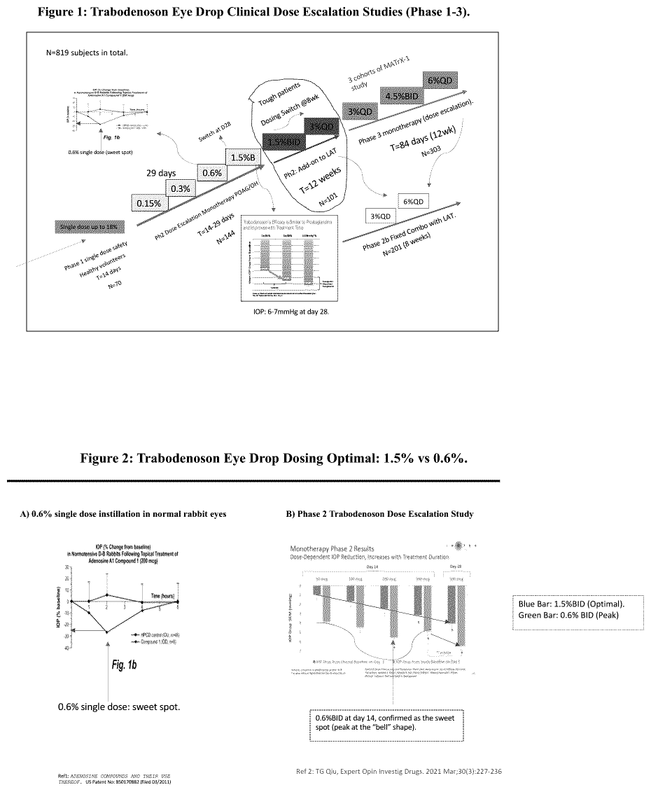

As demonstrated in FIG. 1, Trabodenoson eye drops were previously investigated in randomized controlled clinical trials undergoing dose escalation from Phase 1/2 to Phase 3 in patients with POAG/OH, including a combination study in Latanoprost eye drop poor responders. Table 2 shown in FIG. 1 provides a summary of Trabodenoson (INO-8775) eye drops clinical trials in glaucoma. This clinical trial series validated Trabodenoson derived MMP therapeutic benefit in mild to moderate or advanced glaucoma patients including PGA poor responders, unlike Rhopress, Trabodenoson therapeutic and IOP reduction both arms do not have the up-limit of the baseline IOP<=24 mmHg, Trabodenoson is more effective in patients at high baseline IOP or more severe conditions. The IOP time course improvement is the key pattern behavior of Trabodenoson therapeutic in glaucoma.

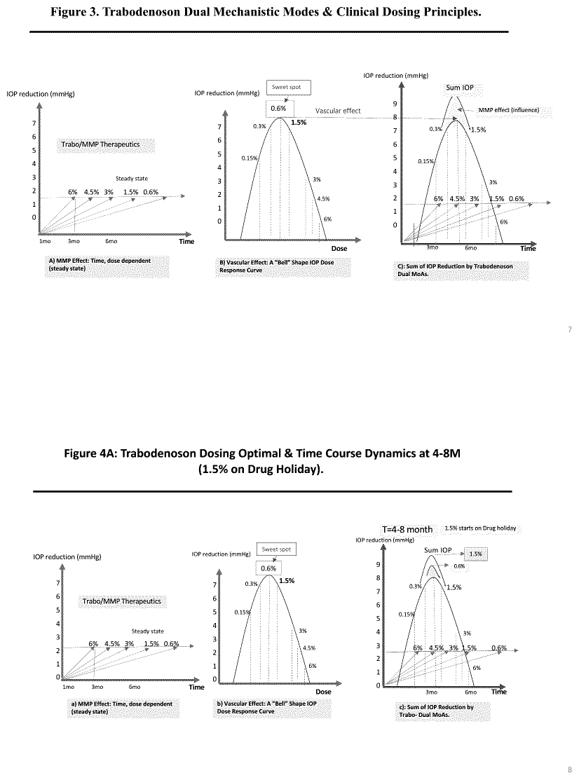

FIG. 2 and FIG. 3 provide detail regarding the proprietary discovery demonstrating optimal dosing at 1.5% vs. 0.6%. Phase 2 Trabodenoson dose escalation study showed a “Bell” shape IOP dose response curve with 0.6% BID peaked at the curve (0.15%, 0.3% 0.6%, 1.5%, 3%, 4.5% and 6%) and a dose dependent IOP reduction with 1.5% BID being more effective than BID at day 14 in normalizing the diurnal variance in patients with glaucoma (0.15%, 0.3%, and 1.5%). 3% is better than 6% on IOP reduction. This is the first time recognizing the two components pertinent to dual MOAs of Trabodenoson given at therapeutic dose (range).

The present disclosure provides for the first time that although Trabodenoson was associated with IOP reduction, the mechanism was not heretofore understood. As discovered by the current inventor, Trabodenoson has a significant influence producing a vascular effect that is the primary driving force of IOP reduction (maximal), whereas MMP effect improves the IOP, does not have a direct action towards the contractible tissues or blood vessels that opens up the mesh pore and increases the outflow facility, instead it removes the debris and alters the tissue texture of TM which improves better compliant of contractible tissue in reducing the IOP. The dual effect with fast mode of vascular effect (contractional) synergizes with its slow mode of action via MMP therapeutic.

The novel discovery of the invention comprises the demonstration that Trabodenoson on vascular effect is dose sensitive: 3% is better than 6% on IOP reduction, 0.6% is better than 1.5% at the start before its MMP therapeutic effects ramp up. This invention also demonstrates that Trabodenoson induces fast mode IOP reduction via vascular effect (which may be NO dependent), thus requires an optimal dose (bell shape). 3% performs better than 6% on IOP reduction (preclinical and clinical evidence). See FIG. 8 which shows that Trabodenoson 3% is better than 6% on the “bell” shape IOP dose reduction curve, a fast mode via vascular effect (FIGS. 8A & 8B).

Some blood vessels in the body are innervated by parasympathetic fibers (e.g. coronary vessels). These nerves release Ach, which binds to muscarinic receptors on the smooth muscle and/or endothelium. MR on vascular effect is through Gi-proteins coupled muscarinic M2 neuro-vascular modulation increasing nitric oxide (NO) effect, similar to the Ach effect on muscle relaxation with increased NO, MR acts through Gi-protein activation coupling with cAMP production that causes vascular relaxation (dilation) with AIR mediated NO production. The targeted receptor is perhaps through M2 parasympathetic nerve innervation on the blood vessel or contractible tissue such as TM smooth muscle cells, and TM endothelium cell which is most sensitive to NO. Brimonidine has opposite effects causing vascular constriction via Norepinephrine (NE) mediated vascular contraction, where the sympathetic nerve innervation is present in the vascular endothelium and/or smooth muscle cells. Preclinical study has demonstrated that Trabodenoson is more potent in rescuing and protecting the RGC and outer layer photoreceptors from apoptosis in comparing with Brimonidine which lacks effects toward the outer layer photoreceptor cells in an ischemia-reperfusion rodent model (W McVicar 2015 ARVO).

The effect of MMPs on Bruch's membrane rejuvenation discovered by Prof John Marshall and its clinical application of 2RT in retinal specialty care provides additional understanding of Trabodenoson derived MMP on TM basement membrane rejuvenation: both Bruch's membrane and the BM at the TM share the common molecular structure and glycoprotein components: fibronectin, collagen IV and laminin. 2RT and SLT also share a similar action through replenishing or regenerating new cells at the adjacent neighboring area (RPE or TM endothelium) that upregulates MMPs, which results in long-term therapeutic benefits up to years with one-time treatment in early stage POAG/OH.

Prior art pharmaceuticals such as Bimatoprost SR intracameral implant (DURYSTA®) upregulates MMP with high concentration API releasing at the target tissue (iris and CB), resulting in drug holiday in 68% patients by single implant, however more than half of the induction patients required medication rescue in 12-24 months (not stable), possibly due to a proportional increase of intraocular inflammation driven by the high level SR API. Bimatoprost, like other prostaglandin analogue drugs, is proinflammatory by nature, (there is FDA label warning on all the commercially available PGA eye drops including the new generation VYZULTA and ROCKLATAN for their risks associated with cystoid macular edema (CME) and increased intraocular inflammation given at low dose eye drop formula). DURYSTA® releases 4400× higher of API to the target tissue compared to Bimatoprost eye drops (0.03% QD) dosed at day 7. Three consecutive implants of DURYSTA® led to 80% patients on drug holiday sustained for 8 months or longer, however such repeated treatments led to the cornea endothelium cell loss (CEL) at an alarming speed (20% loss in 20 months compared to 1% loss per year in normal aging (ref: DURYSTA® Pivotal Ph3 clinical trials: ARMERTIS-1 and ARMERTIS-2). (See FIG. 10)

Drug holiday reflects the state of TM health and a full functional recovery of the pressure sensor in glaucomatous eyes. It reflects the intricate biological competence of pressure sensing cells that can self-regulate the IOP without the need of extra help of symptom relief IOP medications. Drug holiday parameter determination is based on two key aspects. First: Glaucoma disease staging defines the pressure sensor recoverability (estimated: it needs to have minimal 50-70% pressure sensing cell remained in glaucomatous eyes in order to achieve a full functional recovery and a stable drug holiday for 6-12 months or longer. This parameter includes the percentage of patients: 50%-80%, time to start (1-3 month or 3-6 month induction), and the durability of holiday period (3-6 M or 12-24 M or 3-5 years). Second: Therapeutic potency and treatment time. Potency includes MMP potency and neural protection potency, DURYSTA® does not have neural protection role to the pressure sensing cell, plus it increases inflammation and metabolic stress to the pressure sensing cells. Therefore, Trabodenoson is far more potent than PGA in SR such as DURYSTA®. Therapeutic benefits of Trabodenoson increase with treatment time, hence its SR is more effective than the eye drops as drug holiday inducer.

In principle, sustained release Trabodenoson can induce a drug holiday at a faster speed, in higher percentage of patients including more advanced stage of glaucoma (e.g. patients with 3 medications) with a longer duration compared to DURYSTA®. Also, sustained release Trabodenoson drives higher percentage patients on a more durable drug holiday compared to shorter term sustained release format (e.g. 6 M is better than 3 M for severe glaucoma). However, DURYSTA® MMP therapeutic benefit is proportionally increased with its sight threatening side effect to the cornea and TM health, which has not be recognized before, its MMP benefit is being compromised.

According to previous studies as shown in FIG. 11A, drug holiday durability with single dose implant of DURYSTA® SR (4.2 M loading API of 10 ug and 15 ug) has led to 68%, 47% (estimated), 40%, and 28% patients on drug holiday at 6 M, 9 M, 12 M, and 24 M respectively. FIG. 11B provides DURYSTA® API time course induction of drug holiday: DURYSTA® SR single implant with 4.2 M API loaded with bug and 15 ug led to 40% on drug holiday at 12 M, and 12 month API with 3 consecutive implant of bug led to 80% patients on drug holiday. Estimated about 60% patients on drug holiday by 8-month API (1 or 2 consecutive implants). In contrast, FIG. 11C shows Trabodenoson given at low dose eye drops (1.5% BID) led to unique dosing switch from BID to QD as early as day 29 in mild-moderate POAG/OH, and 8 weeks in Latanoprost poor responders. The dosing switch is a clinical biomarker of the TM durability, a precursor of drug holiday.

In an embodiment, provided herein are novel methods and dosing regimens for improving intraocular pressure comprising the administration of a composition comprising Trabodenoson in a dose of 1.0-3.0%, 1.0-2.0% or 1.2-1.5%, 1.5% to a subject in need thereof. The optimal dose (range) results in the peak IOP reduction in a “bell” shape dose response curve, with 0.6% peaking at the very start and 1.5% (or between 1.0%-1.5%) catching up in a later time. In an embodiment, administration of the Trabodenoson composition enables a unique IOP pattern with a continuous IOP improvement towards a steady state. The pattern behavior comprises a slow mode of action in time, dose and disease staging dependent manner. In an embodiment, the invention as described herein is beneficial for subjects having early stage POAG/OH or advanced stage POAG/OH, including PGA poor to non-responders. In an embodiment, the claimed invention is based on therapeutic improvement as opposed to being limited to symptom relief, wherein therapeutic improvement includes, but is not limited to, the improvement or restoration of the trabecular meshwork functionality through double acts of neural protection and BM rejuvenation of pressure sensing cell metabolic competence, hence the benefits to its longevity.

In an embodiment, the invention as claimed normalizes diurnal IOP irregularities, with 1.5% BID eye drop being more effective than 0.6% BID eye drops in patients with glaucoma. In an embodiment, the invention is effective in reversing mild to moderate dysfunction of the sensing cell metabolic activities in early-mid stage glaucoma with mild to moderate TM pathology, in another embodiment, the invention is more effective in stopping the disease progress and treating advanced stage glaucoma with moderate to severe damaged TM, further preventing the need for high risky glaucoma drainage surgeries.

Though not wishing to be bound by the following theory, it is thought that the invention as claimed herein is effective because Trabodenoson simultaneously activates three independent signal pathways via Alit specific binding affinity that triggers G protein coupled muscarinic M2 receptor activation at the TM and CB, cell hyperpolarization at the TM endothelium and MMP14/MMP2 upregulations at the TM. Furthermore, it is thought that at optimal dose ranges, Trabodenoson induces Alit activation leading to neuroprotection or cytoprotection toward the pressure sensing cells at the TM in glaucomatous eyes. Such neuroprotection or cytoprotection is through G-protein coupled cell membrane hyperpolarization inhibits the calcium influx, and restores cell homeostasis at the TM endothelium cell (or pressure sensing cell). The cell hyperpolarization represents a metabolic resting state of the sensing cell with cytoprotection benefit. It is the M2 receptor activation that leads to IOP reduction via contractible tissues in motion, not its derived MMP biological effects, which was previously mis-understood. Its MMP enzymatic degradation helps improve the compliance of contractible tissue in response to pressure sensors' first order of IOP regulation. Such integral functional relations between MMP upregulation and M2 activation at the TM/CB is first time revealed in this invention.

Also, not wishing to be bound by the following theory, it is thought that at optimal dosing, Trabodenoson results in a maximal IOP reduction through M2 receptor activation. Its vascular effect is NO sensitive requiring optimal dose, too (e.g. 0.6%-1.5%, 1.0-1.5%). Trabodenoson derived MMP14/MMP2 upregulation results in basement membrane (BM) texture change with increased elasticity and hydro conductance (similar to MMP on Bruch's membrane rejuvenation). It is also thought that an increased TM hydro-conductance increases the oxygenation and metabolic waste expulsion of the pressure sensing cells at the pathological TM in patients.

In an embodiment, the composition comprising Trabodenoson, for example in the form of (eye drops), is administered once a day, twice a day, three times a day, four times a day, once a week, twice a week, three times a week. In an embodiment, Trabodenoson eye drops 1.5% (or 1.0-1.5%, 1.5-3%) are given twice a day as a loading dose for an initial period of time, and may then be switched to once a day. In certain embodiments, the loading dose period may be determined based on the disease state, early to advanced stage of glaucoma, and some cases the loading dose period may comprise 1-12 weeks, 2-10 weeks, 4 to 8 weeks, and increments therein. The dosing switch from BID to QD is the precursor of drug holiday, it is unique as the result of its potent therapeutic benefit.

As drug holiday stabilizer or maintainer, Trabodenoson eye drop dosing does not require a maximal IOP reduction or strict compliance, e.g. dose range can be 1.5%-3% QD, 3% QD, 1.5% BID, or 3% at frequency of QD, every another day, or 3 times per week. Evening is better than morning dose.

In an embodiment, the composition comprising Trabodenoson is administered as a sustained release (SR) formulation for preventing and treating IOP failure in glaucoma. For maximal IOP therapeutic effect with clinically meaningful IOP reduction, the sustained products, should ideally result in an API concentration in the aqueous humor, that is equivalent to the same API concentration (range) achieved via eye drops at the given optimal dose (range). In an embodiment, the optimal dose (range) of Trabodenoson eye drops determines the API loading dose and release speed. In an embodiment, the sustained release formulations of the invention can be made using polymer-based PLGA and/or PEG biodegradable, or non-biodegradable materials.

In an embodiment, the sustained release formulation is provided in a form wherein the shape, size and dimension is minimally invasive, and/or is administered via an office-based procedure. The form may be an injectable rod, disc, coil, stent and other comparable formats known to those skilled in the art. In an embodiment, one form for an intracameral “rod” can be made to suit for 27G-30G needle with 0.5-1.0 mm in length with biodegradable materials. API lifespan or sustained drug release may comprise a duration between 3-6 month, 6-12, or 12-24 months, or 1-3 months for single implant. In an embodiment, the sustained release formulation can be delivered via various routes via minimally invasive fashion, such as intracameral, intravitreal, suprachoroidal, sub tenon, periocular, ocular surface, puncta plug. The sustained release formulation also can be made via medical coating technology, such as drug eluting stent, drug eluting contact lens, drug eluting intraocular lens.

In order to minimize clinical side effect related to SR formulation, the biodegradable polymer degradation speed and the API release rate should be guided by the timing for both being eliminated from the aqueous system at the same time, or within 2-3 days or maximal 7 days of differences. Care should be taken to avoid API precipitate or an empty implant without API retained in the AC for extended period as seen in Durysta (Ph3 trials). The superiority advantage of Trabodenoson SR AC rod lies at its repeated dosing without stimulating intraocular inflammation, however, the dosing interval must be based on the drug holiday duration for individual eye and the visibility of the implant, which will ensure for a desirable and targeted pharmacokinetic API release profile.

The novel methods and dosing regimens for improving intraocular pressure as described herein may address problems caused by intraocular pressure as associated with glaucoma. The glaucoma may be early, mid or, advanced, late stage glaucoma. In certain embodiments, the glaucoma is POAG/OH, refractory POAG/OH, advanced stage POAG/OH, end stage POAG/OH, primary open angle glaucoma (POAG), Primary Angle Closure Glaucoma (PACG), normal tension glaucoma, angle-closure glaucoma, uveitic glaucoma, or pseudo exfoliation glaucoma, steroid induced IOP elevation or glaucoma or congenital glaucoma.

In certain embodiments, the administration of the composition comprising Trabodenoson is combined with additional therapeutic intervention. The additional therapeutic intervention may comprise surgical intervention or laser treatment. In certain embodiments, the additional intervention comprises administration of a pharmaceutical agent selected from the group consisting of Xalatan® latanoprost, Lumigan®, bimatoprost, Travatan Z®, Travoprost, Zioptan™, tafluprost, Vyzulta™ (latanoprostene bunod), beta blockers, timolol, alpha agonists, Alphagan®P, brimonidine, Iopidine®, carbonic anhydrase inhibitors, Trusopt®, dorzolamide, Azopt®, brinzolamide, Diamox, acetazolamide, Neptazane® (methazolamide), Rho khinase inhibitors, Rhopressa®, Rocklatan®, netarsudil or Cosopt®, Omlonti, or Ripasudil. In certain embodiments, Trabodenoson improves the IOP performance of Rhopressa®, Rocklatan®, Omlonti, or Ripasudil in an advanced or end stage glaucoma. In certain embodiments, the administration of the composition comprising Trabodenoson takes place following a trabeculectomy or tube shunt in advanced of late stage glaucoma, following administration of SLT, Ellios laser or transscleral laser. In certain embodiments, the administration of the composition comprising Trabodenoson takes place following MIGS implant with/without cataract removal, MIGS device may comprise a stent, Hydrus, microshunt, trabectome, istent infinity, iDose in early-moderate and refractory glaucoma respectively. Trabodenoson eye drop is a desirable solution for stabilizing and prolonging the drug holiday in patient receiving Durysta with unstable holiday (e.g. less than 2 months).

In an embodiment, the invention herein comprises methods for repairing the pressure sensor in an eye comprising the administration of a composition comprising Trabodenoson in a dose of in a dose of 1.0-2.0%, 1.0-1.5% or 1.5% or 3% to a subject in need thereof. In an embodiment, the administration of the composition results in MMP14 upregulation, cytoprotection of pressure sensing cells, anti-inflammatory action on/repair of the trabecular meshwork, vascular dilation or neuromodulation. In an embodiment, repair of the TM comprises a reduction of inflammation, a reduction of extracellular matrix, restoration of trabecular meshwork elasticity, and/or reduction of mesh pore blockage. Rejuvenate and repair of the TM, restores the functionality of the pressure sensor in glaucomatous eyes and leads to a drug holiday status allowing for prolonged suspension of IOP reducing treatment.

In an embodiment, glaucoma patient drug holiday can be induced by either Trabodenoson eye drops and/or its sustained release formulation product. In an embodiment, Trabodenoson is provided via a sustained-release biodegradable AC rod that can be used as a loading dose delivery of MMP-14 therapeutic to quickly induce drug holiday. The drug holiday may be as early as within 3 months in early stage glaucoma, with a duration up to 3 years. Such Trabodenoson AC rods may induce a drug holiday in treated patients or refractory glaucoma, estimated with 1-3 month, 3-6 month or 6-12 month API presence (preferable 2-4 months).

In certain embodiments, the Trabodenoson AC rod may be inserted on a periodic basis to a given eye, e.g. every 3-6 months, or 6-9 months, 9-12 months, or every year, or every two years, or every three years or less frequently, depending upon the disease staging and the API sustained release duration. Furthermore, Trabodenoson eye drops may be used as drug holiday inducer with a fixed BID dosing regimen as a monotherapy or combination with current standard care of eye drops or MIGS. In certain embodiments, Trabodenoson eye drops are used as a “relay” of drug holiday inducer following the MMP loading dose delivered by Trabodenoson AC rod or DURYSTA®. The eye drops can be used as drug holiday inducer with a fixed BID dosing regimen as a monotherapy or combination with MIGS, or current standard care of eye drops. Once patient reaches to a drug holiday by SR MMP or MMP drops, there is no requirement of IOP control or reduction at which point Trabodenoson dosing and regiment can be more flexible e.g. 3% QD may be preferable. In an embodiment, Trabodenoson eye drops with higher dose (e.g. 1.5%-3%) can be used as drug holiday maintainer or stabilizer with a flexible dosing regimen (BID, QD or every-another day, or 3 times a week, no strict compliance required.

Trabodenoson eye drop (1.5%, or 1.0-1.5%) has superior advantage for treating previously untreatable or refractory IOP elevation in conditions involving severe intraocular inflammation such as uvetic glaucoma, pseudo exfoliation glaucoma, steroid induced IOP elevation or glaucoma in patients with intraocular implant of sustained release steroids, such as Iluvein or Ozurdex implant or YUTIQ®. Those patients have vision threatening ocular comorbidities such as diabetic retinopathy or diabetic macular edema, retinal vein occlusion, pan-uveitis etc.

Trabodenoson SR AC rod has superior advantage for treating an advanced to late stage glaucoma including but not limited to POAG/OH patients, because of its superior therapeutic potency and excellent clinical safety that falls short in Durysta or other PGA related SR intraocular implants. Trabodenoson SR AC rod may provide 24/7 MMP therapeutic recovery of a pathological TM before the pressure sensing cell death reaches its threshold of cell loss (e.g. >50% loss) toward an irreversible stage. The speed of recovery without safety compromise is the essence to this group of glaucoma patients.

Optimal Trabodenoson Dosing, Unique Molecular Traits and Unique IOP Pattern Behaviors in Patients with Glaucoma.

Trabodenoson is adenosine mimetic and specifically binds to A1 receptor (A1R). It is the first IOP “therapeutic” agent with the ability to repair and restore the pressure sensor functionality in patients with glaucoma. At the same time, it can effectively lower the IOP at 6-7 mmHg at day 28 in patients at early stage POAG/OH, with possible incremental increase of its IOP reduction over time (unique pattern). Given at optimal dose or dose range (specifically, an optimal API concentration at the aqueous humor), Trabodenoson can simultaneously stimulate three independent signaling pathways via G-protein coupled cell membrane hyperpolarization (a resting stage), MMP14 and MMP-2 upregulation at the TM endothelium, and muscarinic receptor 2 activation at the ciliary body and TM (similar to pilocarpine on M3 activation but without miosis).

In particular, Trabodenoson's derived MMP-14 biological effect leads to TM texture changes with increased tissue elasticity and hydro conductance that is similar to MMP-2 driven Bruch's membrane rejuvenation in the retina. Bruch's membrane is a type of the basement membrane (BM) and shares similar molecular structure to the BM that is made of collagen IV, laminin, and fibronectin. The BM health depends on a constant ECM remodeling by an appropriate ratio of MMP and TIMP (tissue inhibitor of metalloproteinases), with aging MMP downregulated, ECM degradation slows down causing thickening of the BM and clotting the mesh pore, therefore the TM tissue becomes rigid and loses its elastic youth and hydro conductance. Trabodenoson triggers MMP-14 upregulation that accelerates the ECM turnover, cleans up the mesh pore and increases the hydro conductance of the BM which further improves oxygenation through the BM/ECM to the pressure sensing cells. Along with its cytoprotection properties and neural protection to the pressure sensing cell, Trabodenoson synergistically boosts patients' responses to other IOP medications such as ROCK inhibitors, especially at an advanced stage of glaucoma. Due to its dual effects of cyto-protection and BM rejuvenation, Trabodenoson is a more potent therapeutic agent than prior art therapeutics such as DURYSTA® which requires very high API concentration to trigger MMP upregulation (but without MMP14 and MMP2 release). Commercially available PGA eye drops do not upregulate MMPs at the aqueous humor (J. Y. Heo, Y. H. Ooi, D. J. Rhee, 2020).

Trabodenoson derived MMP-14 is membrane type MMP subfamily with ˜66 kDa M it is one of most potent MMP enzymes and it removes various types of ECM debris at the thickening and rigid BM, such as glycoproteins (fibronectin, vitronectin, laminin and tenascin), proteoglycans and GAGs (decorin, versican, hyaluronan [HA]), collagens (types I, III, IV, V and VI), elastic fiber components (MAGP-1, fibrillin) and the glaucoma-associated protein, myocilin which are often seen in human glaucomatous eyes. Its down regulation or deficit is associated with premature aging. Upregulation of MMP-14 is associated with macrophage activation and its derived anti-inflammatory activity in aging tissues, which sets it apart from proinflammatory PGA. Bimatoprost and Travvoprost SR induced MMP upregulation is proportionally increased with a heightening intraocular inflammation, even with single implant, there is a clinically visible or measurable intraocular inflammation observed in the AC such as AC flare, synechia or depigmentation changes round the implant (Ocular Therapeutics and Allergan study reports). PGA requires a high concentration in order to stimulate MMP upregulation, MIMP1, MIMP3, MMP-9 are most reported MMP phenotype in invitro culture TM models. None of the PGA eye drops upregulate MMP. DURYSTA®® even at high dose does not upregulate MMP-14 and MMP-2

### Unique Pattern Behavior Leads to Dosing Switch, a Precursor of Drug Holiday.

The low dose of Trabodenoson eye drops (1.5%) comprises therapeutic potency for improving the durability of the pressure sensor, evidenced by the dosing switch from BID to QD as early as seen around day 29, which is unique to Trabodenoson. Such dosing switch is seen in early stage and advanced glaucoma including PGA poor responders. Its derived MMP therapeutic and cytoprotection enables continuous IOP reduction (or improvement) in a time, dose and disease staging dependent fashion. This is the unique IOP lowering pattern behavior, it is the unique identity of Trabodenoson as an IOP drug and driven by its unique MOA and molecular traits, differs from Vyzulta and Rescula or Rhopressa with very limited therapeutic benefit to patients. Such IOP pattern behavior is robust, reproducible and repeatable across early and advanced stages of glaucoma. Such continuous IOP improvement enables dosing switch, which is the first time reported as a precursor of the drug holiday, seen in DURYSTA® sustained release (SR) MMP therapeutic benefit in patients with glaucoma.

### The IOP Lowering Profile of Trabodenoson Differs Between Therapeutic Vs Symptom Relief Drugs

Trabondenoson's unique IOP pattern behavior with a time course improvement is based on a therapeutic profile as the result of a perfect match of drug MoA and disease pathological root causes associated with elevated or uncontrolled IOP or IOP failure. Such therapeutic pattern behavior is driven by its unique molecular traits and dosing regimen. In contrast, most therapeutics available for treating glaucoma (including RHOPRESSA®, VYZULTA® and other IOP lowering drugs are directed to IOP symptom relief, demonstrating a flat IOP reduction profile in a time course fashion e.g. IOP reduction at week 1 and week 12 are the same and consistent for the same group or stage of patients. Vyzulta has a NO donating arm which demonstrated a mild improvement (less than 1.0 mmHg) during the first 12-week, then flattening out in 12-month study in normal tension glaucoma (NTG) in Japanese population. NTG is considered a subset of POAG with IOP independent risk factors.

Trabodenoson is particularly desirable because of its therapeutic effects: not only does it remove the ECM debris via MMP biological effect but it also has unique effect of cytoprotection by restoring the TM endothelium homeostasis, which synergically, improves the pressure sensor's longevity. It applies to all stages of glaucoma (early, mid, late or advanced stage), all types of glaucoma and conditions involving ECM thickening and rigidity at the TM, such as POAG, NTG, PACG, secondary glaucoma, uveitic glaucoma, steroid induced IOP elevation or glaucoma, refractory glaucoma, congenital glaucoma etc.

### Trabodenoson Dosing Principle and Optimal Dose Confirmation: 1.5% Vs 0.6%.

The dosing principle of Trabodenoson compositions is governed by a fast vs slow mode of actions, the fast mode of action results in a “bell” shape dose response curve that requires an optimal dose in order to achieve the maximal IOP reduction (FIG. 3). Single dose instillation of 0.6% Trabodenoson peaks at the “bell” shape IOP dose response curve.

This fast mode of action is of vascular effect, through MR activation induced G-protein coupled M2 receptor activation at the contractible tissue of the CB or TM, similar to pilocarpine on M3 receptor activation leading to IOP reduction but with no miosis. Like most IOP lowering drugs with symptom relief, the fast mode of action synchronizes with the pulsatile heartbeat, similar to blood pressure, IOP is also pulsatile with circadian rhythm and diurnal variances or fluctuations. Trabodenoson or INO-8775 reduces the heart rate in rat via a quick increase of potassium conductance in atrioventricular (AV)-node (resting stage). Ref: Mor, Michal E. et al 20:13.

In glaucoma, patients suffer a greater diurnal variance than normal healthy individual without glaucoma or OH. Such IOP diurnal irregularities in glaucomatous eye are caused by pressure sensor functional irregularities; as glaucoma disease advances from early to mid, and toward end stage, the functional performance of the pressure sensor worsens over time, if the sensing cell loss reaches to a threshold (unknown, e.g. 70% loss around the circle), at which point patients become non-responders and invasive drainage surgeries become inevitable. Similar to cornea endothelium loss that reaches to a threshold (500-1000 cells/mm2) resulting in cornea edema or decompensation and blurring vision, in the TM, there is a threshold of pressure sensing cell loss (edemas or vacuous) before the outflow engine is completely shut down (irreversible damage) and patient becomes medication non-responders. In normal aging process, TM endothelium loss at 0.58% per year per eye (similar to cornea endothelium loss at 0.6% per year in normal physiological condition). In glaucomatous eyes, such process is accelerated, intraocular inflammation and metabolic stress accelerates cell apoptosis. At age 20-year-old, there are about 750,000 cells, at age 80-year-old, there are about 400,000 cells. E.g. For a 60-year-old patient with glaucoma, it's estimated 70% TM cells average at his age maybe required in order to achieve a full recovery or drug holiday.

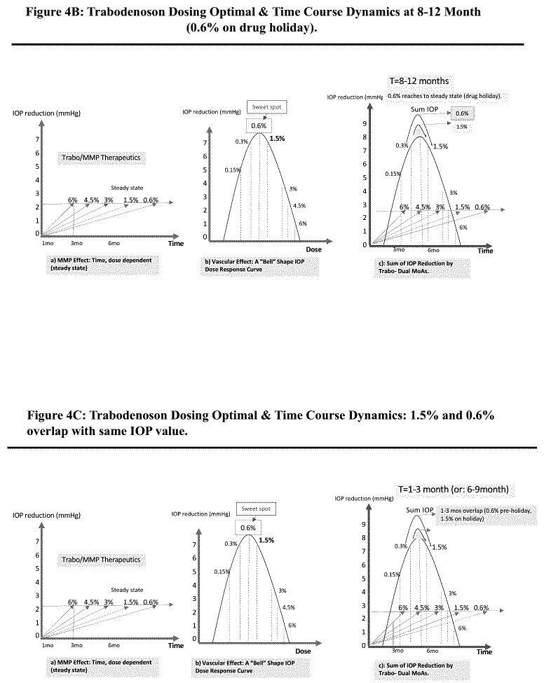

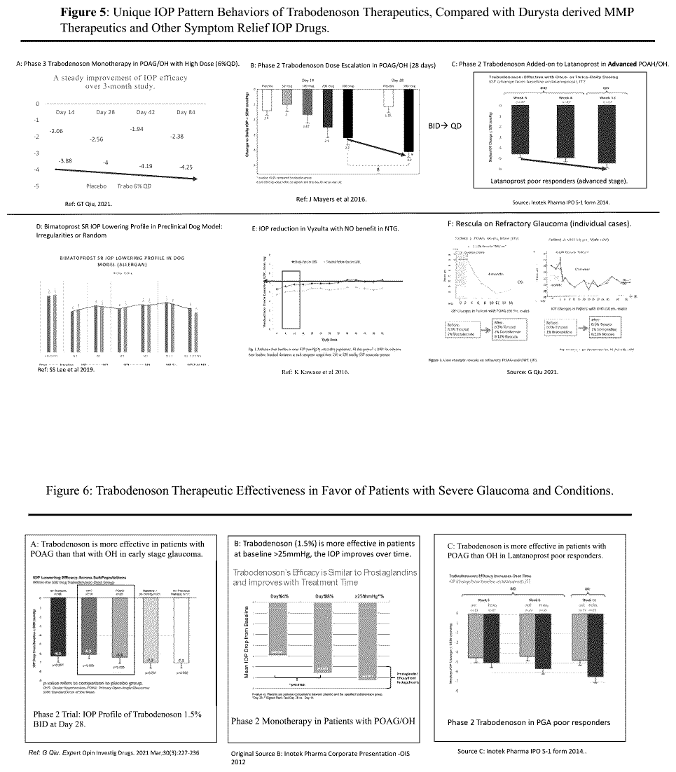

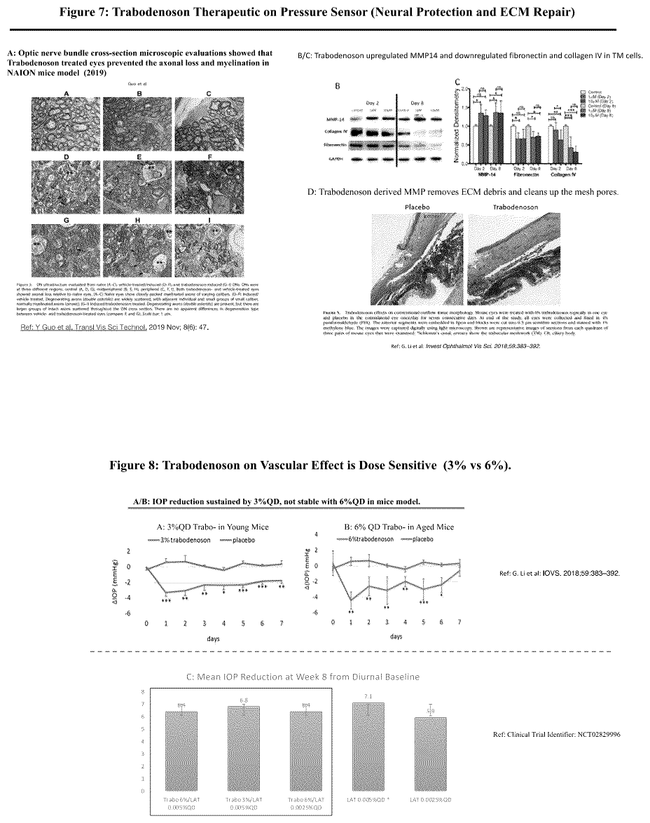

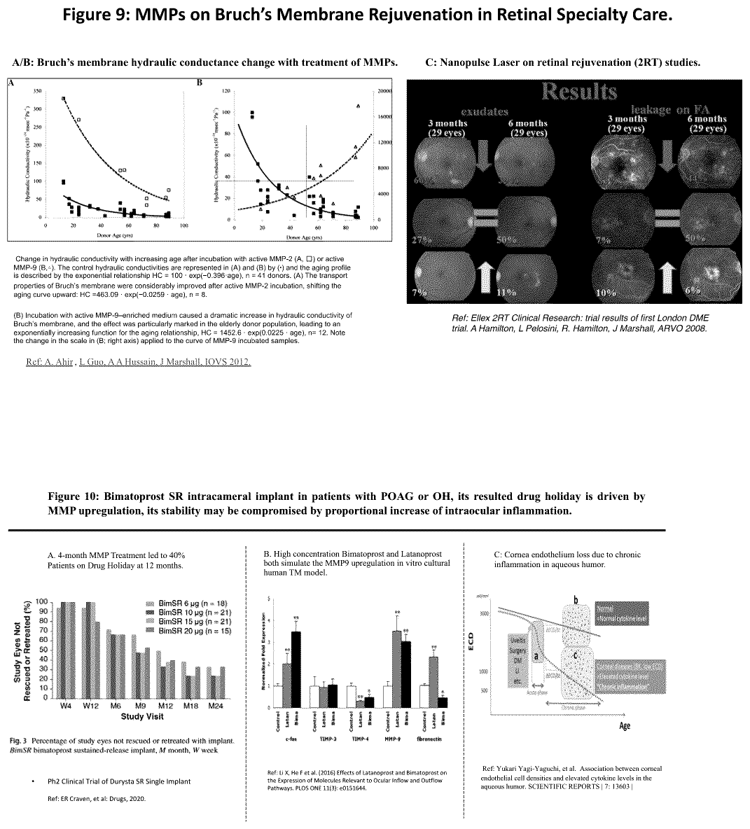

The slow mode of action is the therapeutic arm for Trabodenoson, which demonstrates a time, dose and disease staging dependent manner with a steady state (FIG. 3A). In clinical studies, Trabodenoson eye drops take more than 3 months before reaching to a steady state in early stage POAG/OH with very mild damage. 0.6% has been previously identified as the sweet spot peaking at the “bell” shape dose response curve at the start. However, it was surprisingly discovered that under the positive influence of its MMP therapeutic and its neural protection to the pressure sensor, a higher dose, such as 1.5% BID (or between 1.0-1.5%) is more effective than 0.6% in normalizing the diurnal IOP irregularities and leading to a drug holiday at an earlier time point. Higher dose (1.5% BID) is also more effective in treating severe disease condition that requires a timely repair to avoid massive irreversible damage (cell apoptosis) to the pressure sensor. FIG. 4A/B/C showed three different scenarios of 0.6% BID and 1.5% BID (example of higher dose) interplay tracing toward the finishing line: the steady state.

An optimal dose not only ensures its IOP reduction at the peak of “bell” shape dose response curve, but also requires for achieving a maximal or optimal “therapeutic” benefits through G protein coupled vascular dilation or muscular relaxation that releases nitro oxide (NO).

### Trabodenoson Eye Drop's Dosing & Regimen

As claimed herein, the present invention comprises the administration of Trabodenoson at an optimal dose range of approximately 0.15%-6%, 0.6%-6%, or 3-6%, 1.0-2.0%, 1.5-3%, or preferably 1.0-1.5%, 1.2-1.5%, or specifically, 1.2% or 1.5%.

In an embodiment, the dosing regimen is BID at the start for monotherapy, it can be switched to QD at week 2-4, or week 4-8, or week 8-12, or between 3-6 months, or 6-9 months, or 12-24 months, depending on the needs, disease staging, e.g. drug holiday inducer. The regimen requires BID fixed dose regimen at the start. It is recommended to stay on the drug until drug holiday is achieved, though it can be switched to QD at above mentioned time-points. For drug holiday maintainer or add-on to MIGS or other glaucoma surgeries or laser treatment in patients with their IOP under a control, Trabodenoson eye drop dosing regimen can be flexible at BID, or QD, or three to five times every week, no compliance required. Depending on its clinical utility or purpose, for drug holiday stabilizer or maintainer, the clinical effective dose range of Trabodenoson can be expanded to 3% QD from 1.5% QD, as appropriate.

In an embodiment, for at least one Trabodenoson eye drop formulation: The concentration of Trabodenoson will be the same with 1.5% optimal (or 1.2%, or between 1.0-1.5%, or between 1.0-0.02%, or between 1.5%-3.0%, or 3-6%) in preclinical and all stages of the clinical trials. The eye drops can be made in nanoparticle eye drop formats, microparticle suspensions or emulsions. The formulation may further comprise dextran, or fluorinate or other lipid materials. The eye drops can be made in water-based solutions, lipid-based emulsion or other suspension formulation. The eye drops may or may not comprise preservatives.

### Trabodenoson on IOP Pressure Sensor's Longevity and the TM Health in Glaucoma.

The Components of TM Pressure sensor: The Trabecular meshwork is a porous sponge-alike avascular tissue which is made of ECM contractable tissues ending at the basement membrane and its overlying endothelia cells forming a monolayer lining at the inner wall of Schlemm's canal. For decades, the state of art pharmacological research and drug development has been focusing on the Juxtacanalicular (JCT) region where outflow resistance resides, Rho Kinase inhibitor (ROCK inhibitor) such as Rhopressa is of such drug that relaxes the JCT through regulation of the dynamics of actin fiber (cytoskeleton) of the contractible tissue at the TM, which loosens up the ECM space at the JCT region and resulted in an increased outflow via the TM. Few has realized such contractible activities governed by the pressure sensor, the first order of the command. If the pressure sensing cells are under extreme oxidative stress around the clock even without significant cell loss, Rhopressa won't work, patients become non-responders. Whereas Trabodenoson has the ability to repair and rejuvenate these metabolically compromised cells before they undergo an irreversible cell apoptosis. For example, patients with Hydrus intraocular stent implant with ¼ of the pressure sensing cell being compromised metabolically or dead, Trabodenoson has the ability to protect and prevent the remaining ¾ of the cells from apoptosis as the disease advances toward a later stage, whereas current IOP agents lack of the right MOAs to rejuvenate, repair and restore the functionality of these sensing cells.

The inventor herein is the first to demonstrate that the IOP regulation that lies at the pressure sensor at the end of TM, which is composed of TM endothelium and its underlying BM, is based on Trabodenoson MR target site of action that results in IOP improvement in patients. Like blood pressure that has a pressure sensor at the heart, IOP also has pressure sensor at the TM. It includes two components; one is the pressure sensing cell that is the cobblestone like, neuronal lineage cell, forming a monolayer lining at the inner wall of the Schlemm's canal (sometimes it refers to TM endothelium or Schlemm's canal endothelia cell). It is a shear stress sensor in maintaining the IOP homeostasis in response to oxidative stress, sensitive to the NO level (Fiona McDonnel et al in 2021). The second component is the basement membrane (BM) underlying the TM endothelium, which is composed of collagen IV, lamina and fibronectin in healthy TM, offers the essential support for the health and longevity of the sensing cell that is lacking self-renew upon injury in adult human eyes, similar to RPE cell health depending upon its underlying Bruch's membrane elastic hydro conductance. MMP2 and MMP-14 are the primary MMPs for TM cells to regulate the IOP homeostasis in response to the mechanic stretch via mTOR pathway (ref: J Bradley et al in 2003).

MMP-14 is a potent enzyme that helps to clean up the mesh pores and removing the ECM debris at the BM through protein degradation of fibronectin, lamina and collagen IV and other pathological proteoglycans depositing at the glaucomatous eyes. such as glycoproteins (fibronectin, vitronectin, laminin and tenascin), proteoglycans and GAGs (decorin, versican, hyaluronan [HA]), collagens (types I, III, IV, V and VI), elastic fiber components (MAGP-1, fibrillin) and the glaucoma-associated protein, myocilin precipitated at the TM sheath and beams, which are often seen in human glaucomatous eyes.

Root Cause of IOP Elevation or Uncontrolled IOP in glaucoma: This invention also first time reveals that the pressure sensor dysfunction, worn out or failure is the rate limit and primary root cause associated with the IOP elevation or uncontrolled IOP in patients with glaucoma, which further informs future therapeutic development for clinical management of IOP in glaucoma. The pressure sensor dysfunction in glaucoma is the result of TM endothelium cell aging and apoptosis along with the underlying BM thickening and rigidity as the result of age-related oxidative stress, metabolic inflammation, or para inflammation, similar to RPE loss and Bruch's membrane aging (thickening or atrophy) as the rate limit of dry AMD. Neurovascular inflammation involves both the front and the back of the eye in glaucomatous vision loss. At the front of the eye, it results in pressure sensing cell apoptosis (advanced stage) or functional irregularities at early stage of the disease. Pressure sensing cells are neural lineage cells that share in common with neuronal cell biology. At the back of the eye, neuroinflammation affects the RGC health causing cell apoptosis. Oxidative stress and metabolic inflammation lead to glutamate excitotoxicity in both neuronal cells. Trabodenoson exerts its neural protection through G protein couple cell hyperpolarization which restores the cell homeostasis, hence reducing the oxidative stress or damage to RGC and pressure sensing cells. Trabodenoson has demonstrated in two preclinical model for its neural protection to the RGC apoptosis through potentiating multiple direct RGC protection and indirect neurovascular modulation involving muller glial, astrocyte and microglial, more importantly a potent vascular effect (dilation).

### Trabodenoson & Refractory Glaucoma

Refractory glaucoma represents a clinical state, for which glaucoma patients do not respond well to current symptom relief medications, it could be the result of POAG/OH and PACG at advanced or late stage, or the result of uveitis or PXF associated or steroid induced, or congenital glaucoma. The Trabodenoson therapeutic dosing regimen of the invention provides superior advantage to this group of patients with severe ECM clotting or fibrosis and whose pressure sensor is in dysfunction or damaged, (currently there is no effective treatment available). In POAG/OH, there are about 25-30% patients suffering from inflammatory predisposition with increased proinflammatory molecules such as TNFa, NF-kappa B, Interleukins, bradykinin circulating in the blood stream, some have genetic predisposition such as mutation of optineurin gene (ref. G Qiu 2015). These proinflammatory molecules or cytokines enter into the anterior chamber from the blood stream, which exposes the TM pressure sensor to constant metabolic insults or Para inflammation (a subthreshold inflammation), along with aging related oxidate stress, these risk factors accelerate the sensing cell apoptosis and the thickening of its underlying BM, as well as the fibrotic clotting of the TM mesh pores, collectively it leads to the TM outflow engine broken at the end stage of glaucoma. Such inflamed aqueous humor is often the root cause associated with failures of filtering surgeries as the result of bleb fibrosis. CYPASS® and DURYSTA®® both kill cornea endothelium at an alarming speed in the same subset “inflamed” glaucomatous eyes, for which few may have noticed before.

Uveitic glaucoma involves a more severe intraocular inflammation than advanced POAG/OH, and causes severe damage to the pressure sensor, especially during the acute attack of the disease recurrence. Steroids have been a mainstay treatment for uveitis, however, steroids further deteriorate the TM pathological processes. Most uveitis glaucoma is refractory, and currently there is no effective treatment available.

Pseudoexfoliation syndrome is a systemic disorder in which a fibrillar, proteinaceous substance is produced in abnormally high concentrations within ocular tissues. It is the most common cause of secondary glaucoma worldwide, and the most frequent cause of unilateral glaucoma. The resultant pseudoexfoliation glaucoma responds poorly to medical therapy, compared with other types of glaucoma and can lead to rapid progression of optic nerve damage. The prevalence can reach up to 5% in age of 75-year or older population from 0.6% in age of 52-62 year-old population.

Steroid induced IOP elevation is the result of ECM building up at the TM causing BM thickening, pressure sensor dysfunctional, it is often a transit, most patients can be treated with standard of care IOP lowering drugs, about 4-5% patients may become medication non-responders with recurrent IOP spikes, eventually developing refractory glaucoma that requires high risky glaucoma drainage surgeries, which are often seen in Iluvein and repeated Ozurdex users. MMP therapeutic eye drops is a better option than MMP-based gene therapy for steroid induced IOP spike.

Trabodoenoson eye drops delivering MMP are the optimal choice for steroid induced IOP spikes or glaucoma for broad target populations, including patients with needs resulting from cataract surgeries as well as retinal gene therapy requiring short-term steroid treatment, as well as patients with DME or uveitis requiring a long-term steroid treatment. In contrast, other sustained released products such as DURYSTA®, may result in complications such as heavily inflamed aqueous humor in patients with DME or uveitis that requires long term steroid treatment (e.g. Ozurdex or Iluvein patients).

Primary angle-closure glaucoma (PACG) often has acute onset resulting in synechia or micro fibrosis around the TM in patchy or dotting pattern of damage to the pressure sensor around the clock. To protect the remaining pressure sensor's health and longevity becomes extremely important in patients who either receive trabeculectomy or tube shunt or under standard care of laser or eye drops treatment. The prevalence of PACG is about ⅓ of the POAG population that accounts for 74% glaucoma population, however it is responsible for 50% glaucoma blindness, often seen in a small Asian eye with shallow AC. DURYSTA® implant (27G. 1.0 mm) maybe too big to fit into the small eye with PACG. In contrast, Trabodenoson provides a more potent MMP intervention with neural protection which allows to make a smaller dimension of the “rod” with equivalent slow release duration that could be of an advantage to PACG patients.

Normal tension glaucoma (NTG): NTG is of subset POAG but with patient's IOP under 21 mmHg. Patient visual loss in NTG involves both pressure-independent risk factors such as neurovascular abnormalities and pressure-dependent risks factor with the former being the predominant causative factors, there is no effective treatment available. Trabodenoson offers superiority benefit with overarching therapeutic potential to address the root causes associated with both front and the back of the eye compared to current standard care of medication (symptom relief eye drops).

Trabodenoson therapeutic in combination with current standard of care. For advanced stage glaucoma, Trabodenoson (eye drop or SR) can be combined with the first line PGA drugs, such as Vylzulta, ROCKLATAN, lantanoprost, travoprost, bimatoprost, and second line IOP lowering drugs, such as Rhopressa, timolol, brimonidine, CAI, as “triple” or Combo with 2 medications to manage tough and refractory cases. Preferably, Trabodenoson in combination with ROCK inhibitor such as Rhopressa may improve or booster Rhopressa IOP lowering efficacy via repairing or improving pressure sensor's functionality in this late stage group otherwise medication non-responders. In patients who need 2 to 4 medications, Trabodenoson therapeutic may reverse the disease staging to the point where less medication is required, or stop the disease progress toward the stage, where all the drugs fail and high risky surgeries become inevitable. For advanced or refractory glaucoma, Trabodenoson (eye drop or SR) also can be effective as add-on to patients with MIGS, such as Microshunt, and trabeculectomy, or tube shunt.

### Trabodenoson Therapeutic as a Drug Holiday Inducer or Stabilizer

The term of “drug holiday” was initially used for cancer patient on chemotherapy, during remission period, there is no need for a treatment. In glaucoma IOP management, drug holiday reflects the healthy state of the TM in glaucomatous eye, indicating the pressure sensor's functional recovery to the extend where no medication is required to maintain the IOP at normal or target level. It also reflects the intricate metabolic competence of the pressure sensor that can self-regulate the IOP in response to the shear stretch in patients with glaucoma.

The discovery of Trabodenoson derived MMP leading to a possible drug holiday is based on retinal and glaucoma specialty care with a common thread of the key enzymatic protein, MMP upregulations shared by 1) DURYSTA® derived MMP therapeutic leading to patients on sustained drug holiday, a clinical phenomenon that has not been fully understood before, 2) retinal rejuvenation laser (2RT) induced MMP-2 upregulation in dry AMD patient and the subretinal fluid resolution in patients with DME, which demonstrated sustained clinical benefits for 6-12 months or longer. 3) SLT laser on glaucoma also results in long-term clinical IOP control possible via a similar mechanism to 2RT laser with the MMP upregulation. DURYSTA® derived MMP biological effect has been described by leading experts in the field as a one-time anatomic change causing ECM remodeling, however few realizes that the MMP induced ECM remodeling leads to TM rejuvenation with texture changes of the BM, MMP treatment has the power to turns 60-year-old Bruch's into 40-year-old with increased hydraulic conductance, and helps to regain its elastic youth, ultimately led to better tissue oxygenation and metabolic exchange.

Whether or not a glaucoma patient can achieve drug holiday status and the duration of its drug holiday depends upon the TM recoverability, essentially the quantity and quality of the pressure sensing cell (rate limit) as well as the therapeutic potency and treatment time. For example, Trabodenoson SR is more effective than DURYSTA® SR for a given disease staging, and Trabodenoson SR formula is also more effective than its eye drops in driving patient toward a drug holiday state and repairing severe TM damage such as refractory glaucoma. Single implant of DURYSTA® SR could be potentially more effective than Trabodenoson eye drops (pulse delivery), repeated implant may compromise the pressure sensor functionality due to heightened intraocular inflammation (ARMETIS-1 and 2 Ph3 clinical trials).

The drug holiday induction time and duration directly link to the pressure sensor's recoverability, which is determined by the disease staging or severity and scope of the damage: e.g. 25%, 50%, or 75%, or 80% pressure sensing cell loss (apoptosis) around the Schlemm's canal circle. The therapeutic potency of the agent along with its treatment duration is another important parameter determining the drug holiday induction time and durability. Cell loss and recoverability (unknown): It is not known exactly how many percentages % of the remaining pressure sensing cells required in patients with glaucoma in order to achieve a stable drug holiday for 3 or 6 months or longer, for example. The pressure sensor seems to be very robust metabolically. E.g. 15% patients with Hydrus implant may suffer significant damage of the pressure sensing cells by one quarter (¼) of the circle, with ¾ of the cells remained that can compensate for the ¼ cell loss, some individuals seem still functioning well without the need for extra medication at year-2. Although it is not clear if the 75% remaining cells without Hydrus contact if fully recovered by Trabodenoson MMP treatment would be sufficient to self-regulate, it is estimated that 70% functional sensing cells should be sufficient to compensate for the damage in glaucomatous eyes for a given time if the pressure is under the control.

The quantity and quality of sensing cells are the rate limit of the disease pathological processes related to IOP elevation or uncontrolled IOP, Trabodenoson on TM recoverability is determined by the minimal sensing cell number that can compensate for the loss in chronic and lengthy glaucoma disease progress. Similar to cornea endothelium, there is a threshold of cell loss when the cornea becomes decompensated with edematous change blurring the vision. In normal aging process, TM endothelium loss at 0.58% per year per eye. In glaucomatous eyes, such process is accelerated, intraocular inflammation and metabolic stress accelerates cell apoptosis. At age 20-year-old, there are about 750,000 cells, at age 80-year-old, there are about 400,000 cells. Although there is no established information about the threshold, it is estimated that for a 60-year-old patient with glaucoma, it's estimated 70% TM cells average at his age maybe required in order to achieve a full recovery or drug holiday.

For the same group of patients with POAG/OH under 1-2 meds, as DURYSTA® treated population it is anticipated of at least 80% patients or more in this group that can achieve a stable drug holiday for 6 months or longer with single Trabodenoson SR implant. Specifically, for the same group of patients with POAG/OH under 1-2 meds, it is anticipated that 3-month MMP induction (single implant) could lead to more than 28% patients (e.g. 50%) achieving a stable drug holiday longer than 20-month duration. Specifically, single implant of Trabodenoson with 3-5 month SR API duration, it is estimated 60-70% patients with 1-2 meds who may achieve a stable drug holiday for 6 months up to 3 years. Single implant of Trabodenoson with 1-3 month SR API duration (for smaller eyes), it is estimated 50% patients with 1-2 meds who may achieve a stable drug holiday for 6 months up to 3 years in some individuals. Single implant of Trabodenoson with 3-5 months SR API duration in advanced disease patients with 2-3 meds, MMP induction may lead to 50% patients or more on drug holiday for 6 months or longer. If patients at more advanced stage of glaucoma with 2-3 meds, 3-4 meds or requiring for high risky drainage surgeries such as Tube shunt or Trabeculectomy, Trabodenoson SR implant (AC rod) may reduce the medication from 3-4 to 2-3 meds, or 2-3 meds to 1-2 meds, or replace such invasive risky surgeries that often fails at >50% in 3-5 years. Trabodenoson SR implant (e.g. AC rod) is more effective than its eye drop formula in this advanced stage of glaucoma or refractory glaucoma. Trabodenoson SR implant (e.g. AC rod) has superiority advantage over DURYSTA® in advanced glaucoma (2-4 meds) with its anti-inflammatory and vascular dilation benefits. Trabodenoson can be made in smaller dimension of SR, which can fill the market gaps of primary angle-closure glaucoma for which DURYSTA® is not recommended. DURYSTA® significantly increased intraocular inflammation at an alarming speed causing visual threatening side effects, for clinical safety purpose, only single implant is permitted (limits). Advanced glaucoma patients may be used with cautions.

### Trabodenoson Sustained Release Product Target Profile

In an embodiment, a sustained release device comprising Trabodenoson comprises biodegradable materials (e.g. PLGA or PEG) in a shape suitable for use with an eye, including but not limited to a shape of “rod” and preferable size fitting into a 30G needle (or 27G-30G) and 0.5-1.0 mm in length, via intracameral injection at an office based procedure. In an embodiment, the device should be able to re-dose or repeat at the end of the drug holiday. In an embodiment, the API loading dose and daily release rate or speed is designed in a way that enables API SR duration between 3-6-months which may lead to 80% treated patients achieving a stable drug holiday for 5-6 months at minimal up to 3 years depending on the disease staging: early vs advanced. In an embodiment, for the SR product the API daily release rate (speed) is optimized according to the optimal API concentration in the aqueous humor, determined by eye drops dosing optimal (1.5% BID) at week 4 preferably (between week 1 and week 4). The optimal API concentration in the aqueous humor delivered by Trabodenoson SR or eye drops should be the same with small range of alteration. Such API optimal concentration in the AC or aqueous humor should be the same in preclinical animal models (rabbit, dog, monkey) and in human patients. At such optimal API in the aqueous humor in the AC, Trabodenoson leads to maximal IOP reduction (6-7 mmHg or more) with maximal therapeutic effectiveness.

In certain embodiments, the sustained release Trabodenoson devices envisioned herein comprise devices and formats that can be delivered, administered, implanted or otherwise administered to a patient via intracameral, intravitreal, suprachoroidal, peri ocular, sub tenon, subconjunctiva, retrobulbar, or ocular surface via drug eluting contact lenses or rings, or puncta plug. SR formats can be in various shapes and dimensions, such as rod, disc, stent, rectangular shape. In an embodiment, SR intracameral implant, the size can be made in 27-30G with 0.5-1.0 mm in length. 30G is preferable. SR formats can be made via medical coating technology, e.g. iDose drug eluting stent, drug eluting contact lens, drug eluting intraocular lens (Spyware) and the like.

According to certain embodiments, the Trabodenoson eye drops induced IOP profile has a slow onset with a ramping up time around 4 weeks to achieve 6-7 mmHg, and may take 3 months or longer before it reaches its steady state where maximal IOP reduction is achieved in early-mid stage glaucoma. The Trabodenoson SR AC rod induced IOP profile is estimated of a fast onset with shorter ramping up time than 4 weeks that requires for its eye drop because the MMP exposure continuous. Trabodenoson eye drops deliver MMP twice daily and will take longer time to induce a drug holiday for the same group patients, compared to DURYSTA® MMP and Trabodenoson SR AC rod which delivers the MMP continuously. Although there is no direct reference of eye drop MMP induction time for achieving a drug holiday, based on Trabodenoson eye drops MMP potency with dosing switch from BID to QD occurred as early as 29 days. If using MMP loading dose delivered by DURYSTA® (currently available), it is estimated that within 12-24 months of BID dosing, 50-80% patients with 1-2 meds may achieve a stable drug holiday for 4-6 months or longer. Trabodenoson eye drops are most suitable for being used as drug holiday inducer or stabilizer in combination with a SR MMP delivery by DURYSTA® or Trabodenoson AC rod, patients who have not achieved a drug holiday status, can be continuously induced by MMP therapy by Trabodenoson eye drops, patients who have achieved a drug holiday, Trabodenoson eye drops can be used as a maintainer or stabilizer, within 12-24 months, it is estimated of 50% to 80% patients in total who may achieve a stable drug holiday for 6 months or longer.

Trabodenoson is far more potent MMP stimulator with MMP-14 being the most potent MMP in the family, also it has direct cell protection to the pressure sensing cells, therefore, a Trabodenoson “therapeutic” achieves a longer term drug holiday in more severe or late stage glaucoma patients with the same API induction time. Trabodenoson has excellent clinical safety profile, it is the only drug holiday inducer that does not increase intraocular inflammation.

The sustained release (SR) derived API dose concentration in the aqueous humor should be the same as the API concentration delivered by its eye drops during the first 30 seconds following instillation after multi-dose BID treatment for 4-6 weeks, regardless of the delivery routes, and product prototype materials or SR duration. The API loading dose and release speed (daily) are determined by the API concentration at the aqueous humor where its site of action takes place in the AC tissues (TM and CB). The optimal dose ensures for the maximal therapeutic outcome at the same time the IOP reduction reaches to its maximal steady state between 3-12 months or 6-12 month, or 3-6 months, or 1-3 month.

Trabodenoson Therapeutic on the Back of the Eyes-Trabodenoson & Wet Age-Related Macular Degeneration (AMD): Trabodensoon on Reducing Atrophic Lesions in Patient with Wet AMD Following Long-Term Anti-VEGF Therapy.

Trabodenoson was initially developed for treating cardiovascular disease, pain and inflammation and diabetic, and there are many Ph1-3 clinical trials based on this Adenosine MR related drug for treating cardiovascular and diabetes. Adenosine MR targeted therapy has also been studied in ischemia-reperfusion models in the brain stroke model and the eye (elevated IOP model). MR is most abundant in neurons in the brain and retina, also presents in microglia, astrocytes, oligodendrocytes and Muller's. MR has high density distribution in the inner retina (RGC cell and nerve bundle) but less in the photoreceptor layers. In at least one optic nerve crush model, Trabodenoson eye drops demonstrated neural protection for both RGC inner layer of the neurons and the outer layer of neurons (photoreceptors) in the ischemia reperfusion model, whereas brimonidine did not protect photoreceptor loss. In NAION mice model which also involves ischemia-reperfusion pathological process, Trabodenoson has been observed as a potent neural protectant for RGC cell rescue. In light damaged rat models that mimic one aspect of AMD disease progress, oxidative damage, Trabodenoson also demonstrated its role in rescuing photoreceptor cell loss, through indirect target, perhaps involving Muller glia driven anti-inflammatory pathway and choroidal vascular modulation or vessel dilation, in which its neurovascular modulation is of important for removing the outer segment shedding during intense light damage. Based on such studies, Trabodenoson is a therapeutic candidate for wet AMD atrophic lesion. The key pathology in wet AMD with Lucentis overkill is choroid ischemia around the macular lesion. Eye drops with a formulation that can carry the API towards the choroidal and outer layer of the retina should be effective in reducing such atrophic lesion, as evidence by Rescula clinical study report in this indication, both share one in common that is neurovascular modulator with high permeability to the sclera tissue.

### EXAMPLES

### Example 1

Trabodenoson Therapeutic for patients at early-mid stage POAG/OH. Trabodenoson sustained release (SR) AC rod will be used as loading dose followed by Trabodenoson eye drops as drug holiday maintainers to those who have achieved a drug holiday, or as a drug holiday inducer for those who have not achieved a drug holiday by the loading dose and need extra MMP to drive the repairing and restoration of the pressure sensor toward a full recovery. The endpoint will be the percentage of patients achieved a drug holiday within 3-6 months (or 1-3 months) by Trabodenoson AC rod alone. Because of its therapeutic potency, its IOP ramping up time in SR will be faster than its eye drop 1.5% BID that takes 4 weeks to achieve 6-7 mmHg reduction; Also, E.g. For 3-6 month Trabodenoson SR duration, it is estimated 80% or more treated patients who may achieve a stable drug holiday for 6 months or longer, and its IOP reduction profile will be equivalent to the 2^{nd} line IOP drugs such as Rhopressa or Latanoprost at 7 mmHg or more compared to diurnal IOP baseline starting at week 2, then week 6 and 12 as clinical IOP endpoint (primary endpoint). The remaining 20% patients who receive SR loading dose of Trabodenoson and are heading towards a drug holiday destiny can be supplemented by Trabodenoson eye drops (1.5% BID), it is estimated within 6-12 months, these patients can reach to a stable drug holiday for 6 months or longer.

Trabodenoson SR duration: According to Durysta preclinical study in a normal dog model, it appears that one-month MMP SR treatment could lead to a steady state. Trabodenoson given SR with 1-3 months (or 3-5 months) could be sufficient to induce a stable drug holiday in 60-70% patients for 3-6 months or longer. It can be repeated in those at advanced stage glaucoma with more severe TM damage, who need longer MMP induction and more than one implants, e.g. two consecutive implants, each for 1-3 months or one implant with 3-5 month API release.

If Trabodenoson AC rod is not available, DURYSTA® can be used as alternative as the MMP loading dose to study Trabodenoson eye drop as drug holiday inducer. The clinical endpoint and study design will be similar to using Trabodenoson SR AC rod mentioned above.

A: Clinical Endpoints for Trabodenoson SR AC rod clinical trials: Primary endpoint will be the percentage of patients who achieve stable drug holiday (3-6 months at minimal) following a single implant with 3-6-month SR duration. Rhopressa eye drops will be used as positive control. Co-primary endpoint will be 3-month IOP reduction profile equivalent at all time points at week 2, 6, 12 on 8 am, 10 am and 2 pm to current 2^{nd} line eye drops such as Rhopressa or timolol eye drops.

B: Clinical trial endpoints for Trabodenoson eye drop as drug holiday inducer: using Durysta or Trabodenoson SR (if available) as MMP loading dose followed by Trabodenoson eye drop (1.5% BID) to study the drug holiday parameters. This will be a superiority study in comparing with the 2^{nd} line IOP eye drops, such as Rhopressa or Timolol. The primary endpoint will be the percentage of patients who achieves a stable drug holiday at 12-month endpoint following the SR loading dose of 4-month Durysta MMP treatment. Secondary endpoint: during drug holiday induction period by Trabodenoson eye drops, the IOP will be measured at week 2, 4, 8, 12 and monthly till drug holiday achieves or 12-month endpoint, all the IOP reduction points need to be maintained (except week 2 considering a slow onset of Trabodenoson eye drop) or in comparable with Timolol or Rhopressa, the 2^{nd} line IOP drops whichever applies.

### Example 2

Trabodenoson Therapeutic for refractory glaucoma (late stage POAG/OH). Trabodenoson (SR or eye drops) will be used as “triple” combination for patients at late stage POAG/OH or refractory glaucoma, triple combination will be Trabodenoson (SR or Drop)+Rhopressa+Brimonidine vs Bimatoprost (SR or drop)+Rhopressa+Brimonidine (positive control) for example.

Clinical Endpoints for Trabodensoon SR AC rod as triple combination vs Durysta: 12-month Trabodenoson derived MMP therapeutic resulted in IOP improvement will be the primary endpoints measured by the percentage of patients who can improve or maintain their respective IOPs and the visual field stability without the need for adding the 4^{th} medication or invasive glaucoma surgeries as the results of the disease progression. Secondary endpoints: Percentage of patients who need less of the medication at 12-month endpoint after SR implant with MMP therapeutics. Secondary endpoints: the IOP reduction at week 2, 4, 8, 12 compared to positive control group (equivalent).

Clinical Trials of Trabodenoson Eye drops+Rhopressa+Brimonidine compared with Bimatoprost+Rhopressa+Brimonidine) in refractory glaucoma in patients who need 3 medications at the entry. The primary endpoints will be at 12-month with 24-month extension to compare the two treatment groups of their percentage of patients who need to add the 4^{th} medication or inevitably require for trabeculectomy or tube shunt because of their VF deteriorate or target IOP changes that prompts such treatment decision. The IOP reduction during the treatment has to meet the criteria of target IOP defined at the entry of the study for each study eye or individual which could be varied. In particular, week 2, 4, 8 and 12 IOP will be measured followed by monthly till month 12 or 24 study endpoints.

Anticipated results: Trabodenoson SR will be more effective than Durysta for repairing the pressure sensor failure and dysfunction in this late stage group, therefore, a significant difference of the IOP improvement should be achieved in a higher percentage patient population at 12 months with the SR formula compared to Durysta SR. In Trabodenoson eye drop triple combination therapy study, Trabodenoson is anticipated to perform better than the positive control arm without Trabodenoson or MMP therapeutic, for which patients' IOP will get worse over time, historically this is the known facts. Within the 12-24 month period, some individuals will progress faster than the others to the point that their respective target IOP will need to lower further by adding 4^{th} medication or giving surgical options. In principle, the IOP improvement in SR Trabodenoson will be a faster than that with Trabodenoson eye drops.

1. A method for preventing or treating intraocular pressure (IOP) failure or uncontrolled IOP in an eye comprising the administration of a composition comprising Trabodenoson in a dose in the range of 1.0-1.5%, 3-6%, 1.0-3.0%, 1.5%, 1.2%.

2. The method claim 1, wherein the given optimal dose (range) results in the peak IOP reduction in a “bell” shape dose response curve, with 0.6% peaking at the very start and 1.5% (or between 1.0%-1.5%) catching up in a later time which is more effective therapeutically.

3. The method of claim 1, wherein administration of the composition results in continuous IOP improvement towards a steady state.

4. The method of claim 1, wherein administration of the composition results in MMP14 upregulation, cytoprotection of pressure sensing cells, anti-inflammatory action on trabecular meshwork, vascular dilation or neuromodulation.

5. The method of claim 1, wherein administration of the composition results in reversing and/or stopping glaucoma disease progress by repairing and rejuvenating the pressure sensor.

6. The method of claim 1, wherein the glaucoma is early, mid or late stage glaucoma, refractory POAG/OH, advanced stage POAG/OH, end stage POAG/OH, mild to moderate POAG/OH, Primary Angle Closure Glaucoma (PACG), normal tension glaucoma, congenital glaucoma, secondary glaucoma such as uveitic glaucoma, or pseudo exfoliation glaucoma, steroid induced IOP elevation or glaucoma, uncontrolled IOP elevation or IOP failure associated with other known or unknown pathological root causes.

7. The method of claim 1, wherein administration of the composition comprising Trabodenoson results in repair of trabecular meshwork in the eye, and wherein repair of the trabecular meshwork comprises a restoration and booster of the chief IOP regulator, pressure sensing cell's metabolic competence that allows to regain its intricate self-regulation of the IOP in response to the shear force stretch, with clinical benefits of reducing dosing frequency such as BID switch to QD, or reducing the need for multiple medications, or replacing high risky invasive surgeries, with an ideal scenario of a drug holiday, reflecting a metabolically healthy state of an aged pressure sensor in glaucomatous eyes or patients with uncontrolled IOP.

8. The method of claim 1, wherein the composition comprising Trabodenoson is administered as eye drops once a day, twice a day, three times a day, four times a day, once a week, twice a week, three times a week.

9. The method of claim 1, wherein administration of the composition results in continuous IOP improvement towards steady state, and wherein IOP improvement is therapeutic and not limited to symptom relief.

10. The method of claim 1, wherein administration of the composition comprising Trabodenoson normalizes the diurnal IOP irregularities, with 1.5% BID eye drop being more effective than 0.6% BID eye drops in patients with glaucoma.

11. The method of claim 1, wherein administration of the composition with optimal dose simultaneously activates three independent signal pathways via Alit specific binding affinity that triggers G protein coupled muscarinic M2 receptor activation at the TM and CB, which regulates the fast mode of IOP reduction at maximal (6-7 mmHg or more) via contractible tissues, and cell hyperpolarization (resting state) at the pressure sensing cells along with the MMP14/MMP2 upregulations at the TM, which synergistically contribute to its unique IOP pattern behavior with superior therapeutic potency.

12. The method of claim 1, wherein administration of the composition upregulates MMP14/MMP2 resulting in basement membrane (BM) texture change with increased hydro conductance and oxygenation to the sensing cell via a slow accumulative mode (from weeks to months) towards a long-term functional improvement or recovery of aging TM.

13. A method of improving intraocular pressure in an eye comprising the administration of a composition comprising Trabodenoson in a dose of 1.5%, 1.2%, or a range between 1.0-1.5%, or 1.0-3.0% to a subject in need thereof, wherein the composition is administered as a sustained release formulation with daily released API concentration at the aqueous humor or target tissue equivalent to the level delivered by its eye drop formula given at optimal therapeutic concentration (e.g. 1.2%-1.5%); and wherein the sustained release formulation is provided in the form of an implantable device and wherein the implantable device comprises a rod, stent, microshunt, or microsurgical glaucoma drainage device, and wherein implantable device is made of polymer-based PLGA and/or PEG biodegradable, or non-biodegradable materials.

14. The method of claim 13, wherein the implantable device is delivered in a minimally invasive manner, such as intracameral, intravitreal, suprachoroidal, sub tenon, periocular, ocular surface, or puncta plug, preferably biodegradable AC rod with superiority advantage of repeated dosing periodically (once a year or every 2 or 3 years) and safely without causing undesirable side effects such as CEL.

15. The method of claim 13, wherein the implantable device comprising a sustained release formulation is made via medical coating technology, such as drug eluting stent, drug eluting contact lens, drug eluting intraocular lens.

16. The method of claim 13, wherein sustained release of the composition comprising Trabodenoson results in a drug holiday that is more stable across a broader disease spectrum and sustains longer with less induction time.

17. The method of claim 13, wherein the administration of the composition comprising Trabodenoson is combined with additional therapeutic intervention.
wherein the additional therapeutic intervention comprises surgical intervention or laser treatment or pharmacological drugs;
and wherein the pharmacological drugs are selected from the group consisting of Xalatan® latanoprost, Lumigan®, bimatoprost, Travatan Z®, Travoprost, and Zioptan™, tafluprost, Vyzulta™ (latanoprostene bunod), beta blockers, timolol, alpha agonists, Alphagan®P, brimonidine, Iopidine®, carbonic anhydrase inhibitors, Trusopt®, dorzolamide, Azopt®, brinzolamide, Diamox, acetazolamide, Neptazane® (methazolamide), Rho khinase inhibitors, Rhopressa®, Rocklatan, netarsudil or Cosopt®.

18. The method of claim 1, wherein the administration of the composition comprising Trabodenoson induces or stabilizes, and extends a drug holiday to a subject having glaucoma given at a dose in the range of 1.0-1.5%, 1.0-3.0%, 1.5%, 1.2% or 3-6% with a flexible dosing regimen and no need for strict compliance during drug holiday period.

19. The method of claim 13, wherein the composition is administered via the implantation of a AC rod, preferably with biodegradable formula, and wherein the implantation of the AC rod induces a drug holiday in 80% or more of treated patients including refractory glaucoma, within 3-6 months and wherein the drug holiday lasts for 3 years.

20. A method for treating choroidal ischemia resulted macular atrophy in wet age-related macular degeneration following long-term anti-VEGF therapy, inherited retinal degeneration including but not limited to retinitis pigmentosa, and NAION, as well as glaucomatous neuropathy, comprising the administration of a composition comprising Trabodenoson in either eye drop formula, preferable nanoparticle or lipid based ocular surface delivery or sustained release intravitreal or suprachoroidal delivery.
